(19) United States
(12) Patent Application Publication
(10) Pub. No.: US 2003/0113263 A1
(43) Pub. Date: Jun. 19, 2003

(54) Methods and reagents for using mammalian melanocortin receptor antagonists to treat cachexia

(75) Inventors: Daniel L. Marks, Portland, OR (US); Roger D. Cone, Oregon City, OR (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Oregon Health and Sciences University, a non-profit organization, Portland, OR

(21) Appl. No.: 10/074,754

(22) Filed: Feb. 13, 2002

Related U.S. Application Data
(60) Provisional application No. 60/268,357, filed on Feb. 13, 2001.

(51) Int. Cl. A61K 49/00; A61K 31/00; G01N 33/567

(52) U.S. Cl. 424/9.2; 435/7.21; 514/1

(57) ABSTRACT

The present invention provides recombinant expression constructs comprising nucleic acid encoding mammalian melanocortin receptors, in particular MC-4 melanocortin receptor, and mammalian cells into which said recombinant expression constructs have been introduced that express functional mammalian MC-4 melanocortin receptors. The invention particularly provides such genetically engineered cells expressing the human MC4-R melanocortin receptor for screening compounds for receptor agonist and antagonist activity. The invention also provides screening methods using genetically engineered cells expressing the human MC-4 melanocortin receptor to specifically detect and identify agonists and antagonists for this melanocortin receptor. Such screening methods are provided identifying compounds with MC-4 melanocortin receptor antagonist activity having the capacity to influence or modify metabolism and feeding behavior, particularly pathological feeding behavior such as illness-induced cachexia.

This application claims priority to U.S. Serial No. 60/268,357, filed Feb. 13, 2001.

This invention was made under grant from the National Institutes of Health grant nos. DK55819, DK51730 and HD07497. The government may have certain rights in this invention.

### BACKGROUND OF THE INVENTION

1. Field of the Invention

The present invention relates to the cloning, expression and functional characterization of mammalian melanocortin MC4 receptor genes. The invention provides nucleic acid encoding mammalian, particularly human MC4 melanocortin receptors, recombinant expression constructs comprising said nucleic acids, and mammalian cells into which said recombinant expression constructs have been introduced, and that express functional mammalian MC4 melanocortin receptors. The invention also provides transgenic animals, most preferably transgenic mice wherein the endogenous murine MC4-R locus is either heterozygously or homozygously disrupted. The invention provides methods for using such genetically-engineered cells and animals to specifically detect and identify MC4-R receptor antagonists. Such screening methods provide a means for identifying compounds with MC4-R melanocortin receptor antagonist activity having the capacity to influence or modify physiological function and animal feeding behavior, particularly pathological feeding behavior such as illness-induced cachexia.

2. Background of the Related Art

Under normal circumstances, animals and humans respond to starvation with a complex neuroendocrine response that ultimately leads to an increase in appetite, a relative sparing of lean body mass and burning of fat stores, and an overall decrease in basal metabolic rate (Webber & Macdonald, 1994, Brit. J. Nutr. 71:437-447; Ahima et al., 1996, Nature 382:250-252). In contrast, in some diseases a devastating pathological state of malnutrition known as cachexia arises, brought about by a synergistic combination of a dramatic decrease in appetite and an increase in metabolic rate and metabolism of both fat and lean body mass, producing a relative wasting of lean body mass (Tisdale, 1997, J. Natl. Cancer Inst. 89: 1763-1773; Inui, 1999, Cancer Res. 59: 4493-4501; Fong et al., 1989, Amer. J. Phys. 256: R659-R665; Bruera, 1997, Brit. Med. J. 315: 1219-1222; Emery, 1999, Nutrition 15: 600-603). This combination is found in a number of disorders including cancer, cystic fibrosis, AIDS, rheumatoid arthritis, and renal failure (Tisdale, 1997, ibid.).

The severity of cachexia in many illnesses may be the primary determining factor in both quality of life, and in eventual mortality (Tisdale, 1997, ibid.; Larkin, 1998, Lancet 351: 1336). Indeed, body mass retention in AIDS patients has a stronger correlation with survival than any other current measure of the disease (Kotler et al., 1989, Amer. J. Clin. Nutr. 50: 444-447). Many different tumor types have been studied and it is a common finding that tumor-bearing animals die from cachexia and exhaustion of metabolic fuels, rather than from metastasis or infection (Svaninger et al., 1987, J. Natl. Cancer Inst. 78: 943-950; Emery, 1999, Nutrition 15: 600-603; Svaninger et al., 1989, Eur. J. Cancer Clin. Oncol. 25: 1295-1302; Emery et al., 1984, Cancer Res. 44: 2779-2784). Cachexia is commonly observed in patients with cancer, particularly in children and elderly individuals (Bruera, 1997, ibid.). The resulting malnutrition and loss of lean body mass reduces the quality of life for the affected individual and compromises recovery by decreasing tolerance to therapy and increasing post-surgical complications (Larkin, 1998, ibid.; Inui, 1999, ibid.).

Attempts at drug therapy for cachexia with a variety of agents has met with limited success (DeConno et al., 1998, Eur. J. Cancer 34: 1705-1709; Windisch et al., 1998, Ann. Pharmacother. 32: 437-445; Rivandeneria et al., 1999, Nutr. Cancer 35: 202-206; McCarthy, 1999, Res. Nurs. Health 22: 380-387). The most widely utilized agent, megestrol acetate, has shown some promise in reversing weight loss, but this is primarily due to increases in fat mass and water retention, rather than preservation of lean body mass (Strang, 1997, Anticancer Res. 17: 657-662).

One model that has been particularly useful in studying cachexia has been administration of a purified product found in the cell wall of gram negative bacteria known generically as lipopolysaccharide (LPS). Early experiments focused on the ability of LPS injections to reliably produce anorexia in experimental animals (Baile et al., 1981, Physiol. Behav. 27: 271-277; Murray & Murray, 1979, Amer. J. Clin. Nutr. 32: 593-596). LPS administration is now established as a recognized model for analysis of appetite and metabolism during the early stages of illness-induced cachexia. In addition, there are a number of murine models of cancer cachexia exist that recapitulate the anorexia, rapid weight loss, and catabolism of body protein stores found in human cancer patients. Subcutaneous injections of Lewis lung adenocarcinoma (LLA) or various types of methylcholanthrene-induced sarcomas reliably produce cachexigenic tumors in mice and therefore provide useful models for genetic and pharmacologic analysis of this disorder and its potential treatment (Matthys et al., 1991, Eur. J. Cancer 27: 182-187; Llovera et al., 1998, Cancer Lett. 130: 19-27; Ushmorov et al., 1999, Cancer Res. 59: 3527-3534; Svaninger et al., 1987,J. Natl. Cancer Inst. 78: 943-950).

Most features of the cachexia observed in prolonged illness can be reproduced by chronic infusion of cytokines (Plata-Salaman, 1998, Psychoneuroendocrinology 24: 25-41; Masotto et al., 1992, Brit. Res. Bull. 28: 161-165; Vallieres et al., 1999, Endocrinology 140: 3890-3903; Sherry et al., FASEB J. 3: 1956-1962; Plata-Salaman et al., 1996, Phys. Behav. 60: 867-875; Fong et al., 1989, ibid.). Numerous previous studies have either suggested or demonstrated that cytokines released during inflammation and malignancy act on the central nervous system to alter the release and function of a number of key neurotransmitters, thereby altering both appetite and metabolic rate (Tisdale, 1997, ibid.; Plata-Salaman, 1989, Brit. Behav. Immun. 3: 193-213; Plata-Salaman, 1994, Amer. J. Physiol. 266: R1711-R1715; Inui, 1999, ibid.). Lipopolysaccharide (LPS) potently stimulates the release of numerous cytokines from immune cells in the periphery and glia within the central nervous system (CNS) and that these cytokines are primarily responsible for the observed physiological response (Hillhouse et al., 1993, Brit. J. Pharmacol. 109: 289-290; Van Dam et al., 1995, Neuroscience 65: 815-826; Cavaillon & 1990, Cytokine 2: 313-329; Molloy et al., 1993, Brit. J. Surg. 80: 289-297). However, the neural systems involved in transducing these complex signals remain poorly defined.

Proopiomelanocortin (POMC) is a propeptide precursor that is produced in neurons found in the hypothalamic arcuate nucleus (Jacobowitz & O'Donohue, 1978, Proc. Natl. Acad. Sci. USA 75: 6300-6304). The POMC gene product is processed to produce a large number of biologically active peptides. Two of these peptides, α-melanocyte stimulating hormone (αMSH), and adrenocorticotropic hormone (ACTH) have well understood roles in control of melanocyte and adrenocortical function, respectively. Both of these hormones are also found in a variety of forms with unknown functions, for example, γ-melanocyte stimulating hormone (γMSH), which has little or no ability to stimulate pigmentation (Ling et al., 1979, Life Sci. 25: 1773-1780; Slominski et al., 1992, Life Sci. 50: 1103-1108). A melanocortin receptor gene specific for each of the αMSH, ACTH and γMSH hormones has been discovered by one of the present inventors (see U.S. Pat. Nos. 5,280,112, 5,532,347 and 5,837,521, incorporated by reference herein). In addition, two other melanocortin receptor genes have been discovered by one of the present inventors (see Lu et al, 1994, Nature 371: 799-802; Mountjoy et al, 1994, Molec. Endocrinol. 8: 1298-1308) and others (see Gantz et al., 1993,J. Biol. Chem. 268:15174-15179 and Labbe et al., 1994, Biochem. 33:4543-4549).

Along with the well-recognized activities of αMSH in melanocytes and ACTH in adrenal and pituitary glands, the melanocortin peptides also have a diverse array of biological activities in other tissues, including the brain and immune system, and bind to specific receptors in these tissues with a distinct pharmacology (see, Hanneman et al., in Peptide Hormone as Prohormones, G. Martinez, ed. (Ellis Horwood Ltd.: Chichester, UK)pp. 53-82; DeWied & Jolles, 1982, Physiol. Rev. 62: 976-1059for reviews). POMC neurons are present in only two regions of the brain, the arcuate nucleus of the hypothalamus, and the nucleus of the solitary tract of the brain stem. Neurons from the arcuate nucleus to a number of hypothalamic nuclei known to be important in feeding behavior, including the paraventricular nucleus, lateral hypothalamic area, and ventromedial hypothalamic nucleus. While previous reports have claimed both stimulatory and inhibitory effects of α-MSH on feeding behavior (see Shimizu et al., 1989, Life Sci. 45: 543-552; Tsujii et al., 1989, Brian Res. Bull. 23: 165-169), knowledge of specific melanocortin receptors, their location within the central nervous system and the necessary pharmacological tools were not sufficiently developed at that time to allow the resolution of this issue.

POMC neurons are thought to provide an important tonic inhibition of food intake and energy storage, primarily via production and release of α-melanocyte stimulating hormone (α-MSH) from the POMC precursor. α-MSH binds to central melanocortin receptors (including the type 4 melanocortin receptor, MC4-R). One of the present inventors has shown that a novel antagonist of the MC-3 and MC-4 melanocortin receptors can substantially increase food consumption in animals engaged in normal or fast-induced feeding behavior (see U.S. Pat. No. 6,100,048, incorporated by reference). This is consistent with expression of both MC-3 and MC-4 receptor mRNAs at these sites in in situ hybridization studies (Roselli-Rehfuss et al., 1993, Proc. Natl. Acad. Sci. USA 90:

8856-8860; Mountjoy et al., 1994, Molec. Endocrinol. 8: 1298-1308). Moreover, the regulation of arcuate nucleus POMC gene expression is consistent with an inhibitory role for POMC in feeding behavior. POMC mRNA levels are decreased following a fast (Bergendahl et al., 1992, Neuroendocrinol.56: 913-920; Brady et al., 1990, Neuroendocrinol. 52: 441-447), and a significant diurnal variation in POMC mRNA levels in the arcuate nucleus is seen in rat, with the nadir occurring around the onset of nighttime feeding at 1800 hrs (Steiner et al., 1994, FASEB J 8: 479-488). Stimulation of the hypothalamic MC4-R produces relative anorexia and increased metabolic rate, even in a relatively starved state. Thus, the experimental evidence indicates that POMC neurons play an important role in tonic inhibition of feeding behavior, wherein obesity results from inhibiting central MC-4 receptor tone by antagonism of central melanocortin receptors in several animal models (agouti, MC-4R knockout, POMC knockout).

Studies of the impact of central melanocortins in transducing signals from cytokines have had mixed results. In studies of cytokine-induced anorexia in mice with disrupted melanocortin signaling (viable obese yellow, A^{vy}/a), an enhanced anorexigenic response to peripheral IL1-β was observed (Shimomura et al., 1991, Eur. J. Pharmacol. 209:15-18; Vergoni et al., 1999, Peptides 20: 509-513). In contrast, a recent study has demonstrated that MC4-RKO mice resist the inhibition of locomotion produced with central IL1-β administration (Tatro et al., 1999, Soc. Neurosci. Abst. 25: 1558). Central administration of melanocortin-4 receptor (MC4-R) agonists can inhibit energy intake, increase energy expenditure (Fan et al., 1997, Nature 385: 165-168; Cowley et al., 1999, Neuron 21: 159-163) and reduce body weight (Stair et al., 1999, Soc. Neurosci. Abst. 25: 619; Patterson et al., 1999, Soc. Neurosci. Abst. 25: 618). In contrast, disruption of melanocortin signaling with antagonist administration or deletion of the MC4-R (MC4-RKO) leads to an increase in feeding and eventually to obesity (Fan et al., 1997, ibid.; Huszar et al., 1997, Cell 88:131-141). Pharmacological studies have demonstrated an acute and chronic effect of central melanocortin peptides on feeding behavior (Fan et al., 1997, ibid.; Seeley et al., 1997, Nature 390: 349; Giraudo et al., 1998, Brain Res. 809: 302-306) and energy expenditure (Fan et al., 1997, ibid.; Patterson et al., 1999, ibid.; Huszar et al., 1997, ibid.) that parallel the alterations observed during the development of cachexia.

Recently, Huang et al. (1999, Amer. J. Physiol. 276: R864-R871) demonstrated a reversal of LPS-induced anorexia in rats treated with a central melanocortin antagonist. Huang et al. investigated the impact of central administration of α-MSH or the melanocortin receptor subtype3/subtype4 antagonist SHU-9119 on LPS-induced anorexia and fever in rats. In this study, the investigators found a significant potentiation of the suppressive effects of LPS on food intake with administration of α-MSH, and a reversal of LPS-induced anorexia with SHU-9119 administration. These same treatments reduced and increased LPS-induced fever, respectively.

Thus, there remains a need in the art to develop compounds that interact with and modulate or inhibit the biochemical activity of mammalian melanocortin receptors, such as agonists and antagonists for treating decreased energy intake and increased energy utilization that characterizes illness-induced cachexia.

### SUMMARY OF THE INVENTION

The present invention provides a biological screening system for identifying and characterizing compounds that are antagonists of mammalian, most preferably human, MC-4 melanocortin receptors. The biological screening system of the invention comprises transformed mammalian cells comprising a recombinant expression construct encoding a mammalian, most preferably human MC-4 melanocortin receptor, and expressing said receptor thereby. The invention provides such transformed mammalian cells expressing a mammalian, most preferably human MC-4 melanocortin receptor. Thus, the invention also provides nucleic acid encoding mammalian, most preferably human MC-4 melanocortin receptors, recombinant expression constructs comprising said nucleic acid, and mammalian cells into which said recombinant expression constructs have been introduced, and that express functional mammalian, most preferably human MC-4 melanocortin receptors. The invention also provides an animal having heterozygous of one or homozygous disruption of both endogenous MC-4 melanocortin receptors, preferably a rodent and most preferably a mouse, and nucleic acids for producing such animals. Such rodents, termed “gene knockout”rodents in the art, are also advantageously provided. Methods for using MC-4 melanocortin receptor-expressing mammalian cells or said MC-4 knockout animals to specifically detect and identify antagonists of mammalian, most preferably human MC-4 melanocortin receptor are also provided. Such screening methods provide a means for identifying compounds having MC-4 melanocortin receptor antagonist activity that is associated with the capacity to influence or modify metabolism and behavior in an animal, preferably feeding behavior and most preferably cachexia.

Thus, the invention provides in a first aspect cells for identifying mammalian, most preferably human MC-4 melanocortin receptor antagonists. Preferably the cells comprise a recombinant expression construct encoding a mammalian, most preferably human MC4 melanocortin receptor. As provided by the invention, the mammalian cells of the invention express the MC4 melanocortin receptor encoded by the recombinant expression construct comprising said cell. In preferred embodiments, the melanocortin receptors encoded by the recombinant expression constructs comprising the transformed mammalian cells is a human MC-4 receptor having a nucleotide sequence and amino acid sequence identified by SEQ ID Nos.: 15 and 16, respectively.

In a second aspect, the invention provides a method for using the recombinant cells expressing a mammalian, most preferably human MC-4 melanocortin receptor to identify and characterize test compounds as MC-4 melanocortin receptor antagonists. In this embodiment, the method provided by the invention comprises the steps of contacting the cells with a test compound to be characterized as an antagonist of a mammalian MC-4 melanocortin receptor and detecting binding of the test compound to each of the mammalian melanocortin receptors by assaying for a metabolite produced in the cells that bind the compound. In a preferred embodiment, the detected metabolite is cAMP.

In a preferred embodiment of this method, the mammalian cells expressing mammalian, most preferably human MC-4 melanocortin receptors further comprises a recombinant expression construct encoding a cyclic AMP responsive element (CRE) transcription factor binding site that is operatively linked to a nucleic acid sequence encoding a protein capable of producing a detectable metabolite. In preferred embodiments, said protein is β-galactosidase, most preferably encoded by a nucleic acid comprising the recombinant expression construct identified as pCRE/β-galactosidase (as disclosed in Chen et al., 1994, Analyt. Biochem. 226: 349-354). As provided by the invention, expression of the protein that produces the detectable metabolite is dependent on binding of the test compound to the MC-4 melanocortin receptor expressed by the recombinant mammalian cell and the intracellular production of cAMP as a result. In this embodiment, cAMP production results in expression of a protein capable of producing a detectable metabolite, the protein most preferably being β-galactosidase. In preferred embodiments, the detectable metabolite absorbs light to produce a colored product. Thus, this embodiment of the invention provides MC-4 melanocortin receptor-expressing cells whereby melanocortin hormone binding results in the production of a colored product in proportion to the extent of cAMP production in the cell as a result of hormone receptor binding.

In another embodiment of this aspect of the invention is provided a method for characterizing a compound as an antagonist of a mammalian, most preferably human MC-4 melanocortin receptor. In this embodiment, the method comprises the steps of contacting the cells with an MC-4 melanocortin receptor agonist in an amount sufficient to produce a detectable amount of a metabolite produced in the cells that bind the agonist, in the presence or absence of a test compound to be characterized as an antagonist of a mammalian melanocortin receptor, and detecting the amount of the metabolite produced in each cell in the panel in the presence of the test compound with the amount of the metabolite produced in each cell in the panel in the absence of the test compound. As provided by the assay, inhibition of the production of the detectable metabolite is used as an indication that the tested compound is a melanocortin receptor antagonist, which is further characterized quantitatively by the extent of said inhibition.

In a preferred embodiment of this method, the mammalian cells expressing mammalian, most preferably human MC-4 melanocortin receptors further comprise a recombinant expression construct encoding a cyclic AMP responsive element (CRE) transcription factor binding site that is operatively linked to a nucleic acid sequence encoding a protein capable of producing a detectable metabolite. In preferred embodiments, said protein is β-galactosidase, most preferably encoded by a nucleic acid comprising the recombinant expression construct identified as pCRE/β-galactosidase. As provided by the invention, expression of the protein that produces the detectable metabolite is dependent on binding of the test compound to the melanocortin receptor expressed by the cells. In preferred embodiments, the detectable metabolite absorbs light to produce a colored product. Thus, this embodiment of the invention provides human MC-4 melanocortin receptor-expressing cells whereby melanocortin hormone binding results in the production of a colored product in proportion to the extent of cAMP production in the cell as a result of hormone receptor binding.

The invention provides melanocortin receptor antagonists identified by the methods and using the recombinant cells of the invention. In preferred embodiments, the antagonist is an antagonist of the mammalian, most preferably human MC-4 melanocortin receptor.

The invention also provides methods for characterizing mammalian melanocortin receptor antagonists for the capacity to modify or influence metabolism and feeding behavior in an animal. In a first aspect, the invention provides a method for characterizing a mammalian, most preferably human MC-4 melanocortin receptor antagonist as a stimulator of feeding behavior in an animal. In one embodiment, the method comprises the steps of providing food to an animal not deprived of food for at least 12 hours, with or without administering to the animal an MC-4 melanocortin receptor antagonist, immediately prior to the onset of darkness or nighttime, and comparing the amount of food eaten by the animal after administration of the MC-4 melanocortin receptor antagonist with the amount of food eaten by the animal without administration of the MC-4 melanocortin receptor antagonist. In a preferred embodiment, the method comprises the step of inducing cachexia in an animal and comparing food intake in the animal with or without administration of an MC-4 melanocortin receptor antagonist. In preferred embodiments, cachexia is induced in the animal by administration of lipopolysaccharide (LPS), or by implanting tumor cells or tumor tissue in the animal.

In additional embodiments of the methods of the invention, recombinant cells of the invention are contacted with a test compound to be characterized as an antagonist of a mammalian MC-4 melanocortin receptor and detecting binding of the test compound to each of the mammalian melanocortin receptors by assaying for a metabolite produced in the cells that bind the compound. In further steps of this embodiment of the methods of the invention, test compounds that are identified as mammalian, most preferably human MC-4 melanocortin receptor antagonists are farther administered to mice that have been treated to produce cachexia. Said test compounds that can inhibit or ameliorate cachexia in the animal are identified when cachexia is inhibited or ameliorated in the wildtype animals.

The invention also provides methods for using MC-4 melanocortin receptor antagonists for modifying feeding behavior in an animal. In a first aspect, the invention provides a method for stimulating feeding in an animal, the method comprising administering to the animal an MC-4 receptor antagonist. In a preferred embodiment, the invention provides a method for treating cachexia in an animal or preventing cachexia in an animal at risk for developing cachexia as a consequence of suffering a cachexia-inducing ailment such as AIDS or cancer. In a preferred embodiment, the antagonists are administered systemically. In additional embodiments, the antagonists are administered intracerebroventricularly.

It is an advantage of the present invention that it provides an in vitro screening method for characterizing compounds having MC-4 melanocortin receptor activities that relate to feeding behavior in animals. Specifically, the invention advantageously provides means and methods for identifying compounds having MC-4 melanocortin receptor antagonist activity that have been associated with either stimulating feeding behavior when administered to an animal, most preferably cachexia. The invention thus provides an economical first step in screening compounds for the capacity to affect feeding behavior, including synthetic, peptidomimetic or organomimetic derivatives of MC-4 melanocortin receptor antagonists as disclosed herein or elsewhere.

Specific preferred embodiments of the present invention will become evident from the following more detailed description of certain preferred embodiments and the claims.

### DESCRIPTION OF THE DRAWINGS

FIGS. 1A and 1B illustrate the nucleotide (SEQ ID No.: 15) and amino acid (SEQ ID No.: 16) sequence of the human melanocortin-4 receptor gene.

FIG. 2 shows a graph of intracellular cAMP accumulation resulting from peptide binding to human melanocortin-4 receptor agonist in human 293 cells transfected with a MC-4 receptor-encoding recombinant expression construct, wherein -¤- represents binding of ACTH₄₋₁₀, -●—represents binding of ACTH₁₋₃₉, -▪—represents binding of NDP-MSH, -∘—represents binding of αMSH, -Δ—represents binding of γ₂-MSH, and -▴—represents binding of des-acetyl αMSH.

FIG. 3 illustrates the structure of the pCRE/β-gal plasmid.

FIG. 4 illustrates the results of the β-galactosidase-coupled, colorimetric melanocortin receptor binding assay using cells expressing the MC-4 melanocortin receptor and contacted with αMSH or a variety of αMSH analogues, wherein -▪—represents binding of αMSH, -▴—represents binding of NDP-MSH, -●—represents binding of SHU9128 (para-Fl substituted), -□—represents binding of SHU9203 (p-Cl substituted), -Δ—represents binding of SHU8914 (p-I substituted), and -∘—represents binding of SHU9119.

FIG. 5 shows the results of classic competition binding assays using the melanocortin analogues SHU9119 and SIHU8914 at the MC-4 melanocortin receptor, wherein -▪—represents binding of NDP-MSH, -Δ—represents binding of SHU8914 (p-I substituted), and -∘—represents binding of SHU9119.

FIG. 6 shows the results of cAMP accumulation experiments for the MC-4 melanocortin receptor. -▪—represents binding of MTII, -▴—represents binding of NDP-MSH and -▾—represents binding of forskolin.

FIGS. 7A through 7D are graphs illustrating the effects of agouti-related peptide (AGRP) administration on lipopolysaccharide (LPS)-induced cachexia. FIG. 7A demonstrates that intracerebroventricular (icv) AGRP injection ameliorates LPS anorexia, with effects seen for as long as 24-36 hours. FIG. 7B shows cumulative normalized food intake after LPS injection. FIG. 7C shows that AGRP administration prevents LPS-induced weight loss. FIG. 7D is a graph showing that tolerance to repeated LPS injection is observed in both groups. (*p<0.001, **p<0.0001)

FIGS. 8A through 8D are graphs showing that MC4 receptor knockout (MC4-RKO) mice resist LPS-induced cachexia and illness behavior. LPS results in a decrease in feeding for approximately 36 hours in wildtype (WT), but not MC4-RKO animals when expressed both as total normalized intake (FIG. 8A) or as a percentage of basal (post i.p. saline) intake (FIG. 8B). FIG. 8C shows that normal nocturnal increase in wheel running activity is observed in LPS-treated MC4-RKO animals (data shown is the average turns/min in five animals, measured for 24 hours, starting at 1700 h with lights out at 1900). FIG. 8D shows that young MC4-RKO mice resist LPS-induced growth failure. (*p<0.05,**p<0.01 vs WT control)

FIG. 8E shows normal release of cortisol by MC-4RKO mice in response to LPS administration.

FIGS. 9A through 9D are graphs showing that AGRP administration prevents cachexia in mice bearing a syngenic sarcoma. FIG. 9A shows that feeding can be restored in animals that have already become hypophagic, with the effect lasting for 2-3 days. Arrows indicate the days of injection of AGRP (2.5 nMoles). (*p<0.01 vs. WT control) FIG. 9B demonstrates that injections given earlier in the course of the disease prevents hypophagia in tumor-bearing animals and produces hyperphagia in sham-implanted controls. FIG. 9C shows that AGRP prevents the tumor-induced carcass weight loss (*p<0.0001), without affecting final tumor mass (shown in FIG. 9D).

FIGS. 10A through 10D shows that MC4-RKO mice resist cachexia due to growth of a syngenic adenocarcinoma. FIG. 10A shows that hypophagia during tumor growth in WT animals is not seen in MC4-RKO animals (ANOVA p<0.0001). FIG. 10B shows parallel changes in water lick counts are also observed (ANOVA p<0.0001). FIG. 10C shows that MC4-RKO animals gained carcass weight while WT animals lost weight (*p<0.05 vs WT), but final tumor mass was not affected (FIG. 10D).

FIG. 10E shows that MC-3R knockout mice were even more susceptible to tumor-induced cachexia than wildtype mice, indicating that the results shown in FIGS. 10A through 10D were specific for MC-4 receptor.

FIGS. 11A and 11B show the results of dual X-ray absorptiometry (DEXA) analysis of body composition in wildtype (+/+) and MC-4 receptor knockout (−/−) mice. FIG. 11A shows differences in lean body mass for tumor-bearing and sham-injected animals. FIG. 11B shows corresponding changed in body fat mass.

FIG. 12 shows the results of indirect calorimetry of LPS-treated wildtype and MC-4 receptor knockout mice.

### DETAILED DESCRIPTION OF THE PREFERRED EMBODIMENTS

The term “MC -4 melanocortin receptor” as used herein reference to proteins having the biological activity of the disclosed human MC-4 melanocortin receptor identified by SEQ ID Nos.: 15 and 16, as well as naturally-occurring and genetically-engineered allelic variations in these sequences.

Cloned nucleic acid provided by the present invention may encode MC-4 receptor protein, particularly an MC-4 receptor protein of any species of origin, including, for example, mouse, rat, rabbit, cat, and human, but preferably the nucleic acid provided by the invention encodes MC-4 receptors of mammalian, most preferably rodent and human, origin.

The production of proteins such as the MC-4 receptors from cloned genes by genetic engineering means is well known in this art. The discussion which follows is accordingly intended as an overview of this field, and is not intended to reflect the full state of the art.

DNA which encodes MC-4 receptors may be obtained, in view of the instant disclosure, by chemical synthesis, by screening reverse transcripts of mRNA from appropriate cells or cell line cultures, by screening genomic libraries from appropriate cells, or by combinations of these procedures, as illustrated below. Screening of mRNA or genomic DNA may be carried out with oligonucleotide probes generated from the MC-4 receptor gene sequence information provided herein. Probes may be labeled with a detectable group such as a fluorescent group, a radioactive atom or a chemiluminescent group in accordance with known procedures and used in conventional hybridization assays, as described in greater detail in the Examples below. In the alternative, MC-4 receptor gene sequences may be obtained by use of the polymerase chain reaction (PCR) procedure, with the PCR oligonucleotide primers being produced from the MC receptor gene sequences provided herein. See U.S. Pat. Nos. 4,683,195 to Mullis et al. and 4,683,202 to Mullis.

MC-4 receptor proteins may be synthesized in host cells transformed with a recombinant expression construct comprising a nucleic acid encoding each of the receptors disclosed herein. Such a recombinant expression construct can also be comprised of a vector that is a replicable DNA as construct. Vectors are used herein either to amplify DNA encoding an MC-3 receptor and/or to express DNA which encodes an MC-4 receptor. For the purposes of this invention, a recombinant expression construct is a replicable DNA construct in which a DNA sequence encoding an MC-4 receptor is operably linked to suitable control sequences capable of effecting the expression of the receptor in a suitable host cell. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants. See, Sambrook et al., 2001, Molecular Cloning: A Laboratory Manual, 3^{rd} ed. (Cold Spring Harbor Press: New York).

Also specifically provided by the invention are reporter expression constructs comprising a nucleic acid encoding a protein capable of expressing a detectable phenotype, such as the production of a detectable reporter molecule, in a cell expressing the construct. Such constructs can be used for producing recombinant mammalian cell lines in which the reporter construct is stably expressed. Most preferably, however, the reporter construct is provided and used to induce transient expression over an experimental period of from about 18 to 96 hrs in which detection of the reporter protein-produced detectable metabolite comprises an assay. Such reporter expression constructs are also provided wherein induction of expression of the reporter construct is controlled by a responsive element operatively linked to the coding sequence of the reporter protein, so that expression is induced only upon proper stimulation of the responsive element. Exemplary of such a responsive element is a cAMP responsive element (CRE), which induces expression of the reporter protein as a result of an increase in intracellular cAMP concentration. The melanocortin receptors are known to couple to G-proteins and thereby activate adenyl cyclase, increasing intracellular levels of cAMP (see Buckley & Ramachandran, 1981, Proc. Natl. Acad. Sci. USA 78: 7431-7435; Grahame-Smith et al., 1967, J. Biol. Chem. 242: 5535-5541; Mertz & Catt, 1991, Proc. Natl. Acad. Sci. USA 88: 8525-8529; Pawalek et al., 1976, Invest. Dermatol. 66: 200-209). In the context of the present invention, such a stimulus is associated with melanocortin receptor binding, so that a reporter construct comprising one or more CREs is induced to express the reporter protein upon binding of a receptor agonist to a MC receptor in a recombinantly transformed mammalian cell. Production and use of such a reporter construct is illustrated below in Example 5.

Vectors useful for practicing the present invention include plasmids, viruses (including phage), retroviruses, and integratable DNA fragments (i.e., fragments integratable into the host genome by homologous recombination). The vector replicates and functions independently of the host genome, or may, in some instances, integrate into the genome itself. Suitable vectors will contain replicon and control sequences which are derived from species compatible with the intended expression host. A preferred vector is the plasmid pcDNA/neo I. Transformed host cells are cells which have been transformed or transfected with recombinant expression constructs made using recombinant DNA techniques and comprising mammalian MC-4 receptor-encoding sequences. Preferred host cells are human 293 cells. Transformed host cells are chosen that ordinarily express functional MC-4 receptor protein introduced using the recombinant expression construct. When expressed, the mammalian MC-4 receptor protein will typically be located in the host cell membrane. See, Sambrook et al., ibid.

Cultures of cells derived from multicellular organisms are a desirable host for recombinant MC-4 receptor protein synthesis. In principal, any higher eukaryotic cell culture is workable, whether from vertebrate or invertebrate culture. However, mammalian cells are preferred, as illustrated in the Examples. Propagation of such cells in cell culture has become a routine procedure. See Tissue Culture, Academic Press, Kruse & Patterson, editors (1973). Examples of useful host cell lines are human 293 cells, VERO and HeLa cells, Chinese hamster ovary (CHO) cell lines, mouse Y1 (subclone OS3), and WI138, BHK, COS-7, CV, and MDCK cell lines. Human 293 cells are preferred.

The invention provides a variety of methods that are screening assays for detecting and characterizing antagonists of melanocortin receptor, most preferably MC4-R receptors. The invention provides an assay, comprising cells expressing mammalian, preferably human MC-4 melanocortin receptor either endogenously or as recombinant mammalian cells heterologously expressing the MC-4 receptor. The invention provides such cells also comprising a detection means for detecting receptor antagonist binding, such as the reporter expression constructs described herein, and using direct binding and competition binding assays as described in the Examples below. In the use of this assay, the MC-4 receptor is assayed for antagonist binding to a test compound and binding compared with known MC-4 receptor antagonists to identify new compounds having receptor binding activity associated with a particular behavioral or physiological effect, most preferably prevention, inhibition or amelioration of cachexia.

The invention provides in vitro assays to characterize MC4-R antagonists as a preliminary and economical step towards developing drugs for use in vivo for treating cachexia and other pathological feeding behavior disorders. Cells expressing mammalian MC-4 receptor proteins made from cloned genes in accordance with the present invention may be used for screening MC-4 receptor binding compounds for antagonist activity. Competitive binding assays are well known in the art and are described in the Examples below. Such assays are useful for drug screening of MC-4 receptor antagonist compounds, as detected in receptor binding assays as described below.

One particular use of such screening assays are for developing drugs and other compounds useful in modifying or changing feeding behavior in mammals. In particular, the methods of the invention are useful for identifying compounds having MC-4 receptor antagonist activity for treating cachexia. The invention provides an assay system, comprising recombinant mammalian cells, heterologously expressing the MC-4 receptor disclosed herein. The invention provides such cells also comprising a detection means for detecting receptor binding and antagonist activity, such as the reporter expression constructs described herein, using direct binding and competition binding assays as described in the Examples below. In the use of these cells, the MC-4 receptor is assayed for antagonist activity of a test compound, compared with known MC-4 receptor agonists and antagonists to identify new compounds having a receptor binding activity associated with a particular behavioral or physiological effect, most preferably inhibition or amelioration of cachexia.

For example, provided herein is experimental evidence that MC-4 receptor antagonists are capable of inhibiting or ameliorating cachexia in experimental animals in three different animal models of cachexia. The invention provides an in vitro assay to characterize MC-4 antagonists as a preliminary and economical step towards developing drugs for treating cachexia in vivo.

The invention also provides an in vivo assay for identifying compounds that inhibit or ameliorate cachexia in an animal. In preferred embodiments the compounds are MC-4 melanocortin receptor antagonists. In the practice of this aspect of the methods of the invention a test compound is administered to an animal with cachexia, and fat or lean body mass loss, or reduction in energy metabolism, food intake or motor activity is compared in animals administered the compound with a cachexic animals that have not been administered the compound. Test compounds are determined to be capable of inhibiting or ameliorating cachexia when less fat or lean body mass loss or reduction in energy metabolism, food intake or motor activity is detected in animals administered the test compound than in animals not administered the test compound. In preferred embodiments, cachexia is induced in the animals by lipopolysaccharide administration or by implantation of tumor cells or tissue in the animal.

The invention also provides methods that combine the in vitro and in vivo assays described herein, whereby a test compound is first assessed in vitro for MC-4 melanocortin receptor antagonist activity and then MC-4 melanocortin antagonists identified thereby are assayed in vivo to detect cachexia inhibiting or ameliorating properties thereof.

The MC-4 receptor binding analogues, in particular those analogues that are MC-4 receptor antagonists are provided to be used in methods of influencing, modifying or changing feeding behavior in mammals in vivo. Specific examples of uses for the MC-4 receptor binding analogues of the invention include but are not limited to treatment of eating disorders such as anorexia and obesity, and other pathological weight and eating-related disorders. In preferred embodiments, the compounds are advantageous for treating failure to thrive disorders and especially disease-related cachexia, such as occurs in cancer patients, cystic fibrosis and AIDS sufferers, and in cases of renal failure. Also preferred are embodiments of these methods for preventing cachexia in an animal at risk for developing cachexia as a consequence of suffering a cachexia-inducing ailment such as AIDS or cancer. Also within the scope of the analogues of the invention is use for enhancing appearance, athletic ability, or adjuvant to other therapies to treat disorders such as high blood pressure, high serum cholesterol, vascular and heart disease, stroke, kidney disease, diabetes and other metabolic disorders.

The invention also provides embodiments of the compounds identified by the methods disclosed herein as pharmaceutical compositions. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

Non-toxic pharmaceutical salts include salts of acids such as hydrochloric, phosphoric, hydrobromic, sulfuric, sulfinic, fornic, toluenesulfonic, methanesulfonic, nitric, benzoic, citric, tartaric, maleic, hydroiodic, alkanoic such as acetic, HOOC—(CH₂)ₙ—CH₃ where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

For injection, MC-4 receptor antagonist compounds identified according to the methods of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, nitic, benzoic, citric, tartaric, maleic, hydroiodic, alkanoic such as acetic, HOOC—(CH₂)ₙ—CH₃ where n is 0-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds of the present invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays, as disclosed herein. For example, a dose can be formulated in animal models to achieve a circulating concentration range that includes the EC50 (effective dose for 50% increase) as determined in cell culture, i.e., the concentration of the test compound which achieves a half-maximal inhibition of bacterial cell growth. Such information can be used to more accurately determine useful doses in humans.

It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician.

Preferred compounds of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B 677: 1-27). Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Dosage amount and interval can be adjusted individually to provide plasma levels of the active moiety that are sufficient to maintain bacterial cell growth inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound cannot be related to plasma concentration.

The disclosures in this application of all articles and references, including patents, are incorporated herein by reference.

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLE 1

### Isolation of a Human MC-4 Receptor DNA

For cloning the human MC-4 receptor, a human genomic library was screened at moderate stringency (40% formamide, 1M NaCl, 50 mM Tris-HCl, pH 7.5, 0.1% sodiumpyrophosphate, 0.2% sodium dodecyl sulfate, 100 μg/ml salmon sperm DNA, 10×Denhardt's solution, 42° C.), using ³²P-labeled rat PCR fragments isolated as follows. Primers as set forth below (commercially available from Research Genetics Inc., Huntsville, Ala.).:

| | | |
|---|---|---|
| Primer II (sense): | GAGTCGACC(A/G)CCCATGTA(C/T)T(AGT)(C/T)TTCATCTG | (SEQ ID NO:3) |
| and | | |
| Primer VII (antisense): | CAGAATTCGGAA(A/G)GC(A/G)TA(G/T)ATGA(A/G)GGGGTC | (SEQ ID NO:4) |

were used in a polymerase chain reaction amplification of rat melanoma cDNA. PCR reactions were performed in 100 μL of a solution containing 50 mM Tris-HCl (pH 8.3), 2.5 mM MgCl₂, 0.01% gelatin, 200 μM each dNTP, and 2.5 units of Taq polymerase (Saiki et al, 1988, Science 239: 487-491). Each PCR amplification cycle consisted of incubations at 94° C. for 1 min (denaturation), 45° C. for 2 min (annealing), and 72° C. for 2 min (extension). Amplified products of the PCR reaction were extracted with phenol/chloroform and precipitated with ethanol. After digestion with EcoRI and SalI, the PCR products were separated on a 1.2% agarose gel. A slice of this gel, corresponding to PCR products of 300 basepairs (bp) in size, was cut out and purified using glass beads and sodium iodide, and the insert was then cloned into a pBKS cloning vector (Stratagene, La Jolla, Calif.).

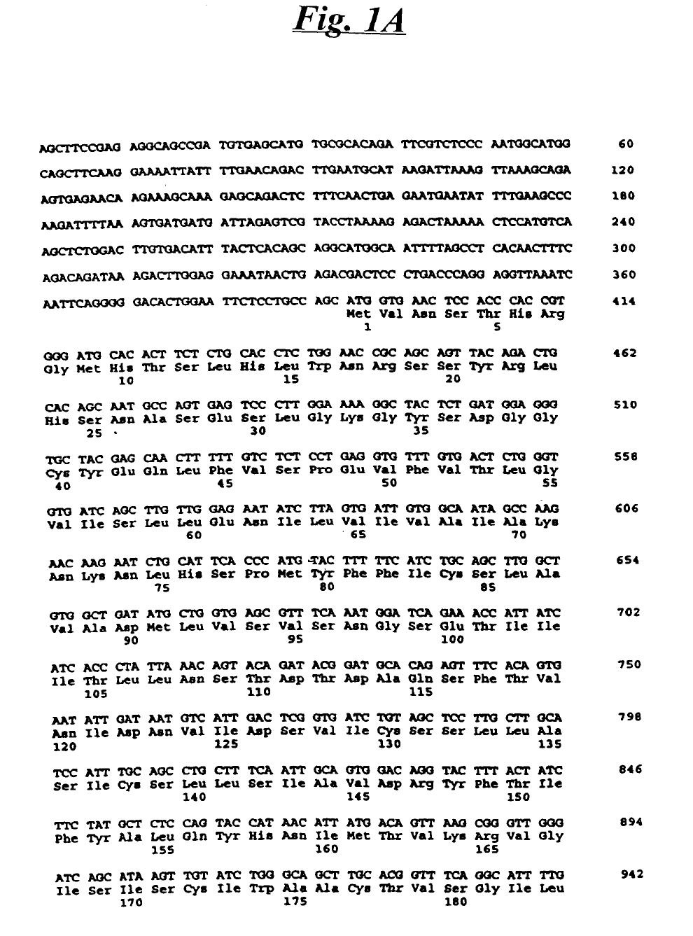

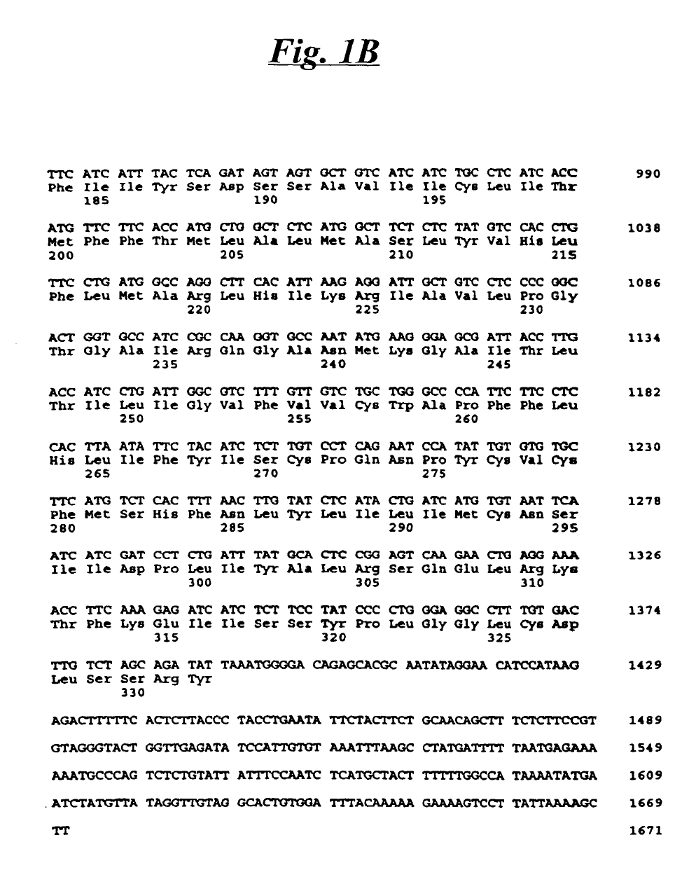

A genomic clone was isolated that encoded a highly related G-coupled receptor protein (SEQ ID NOs: 1 and 2; shown in FIGS. 1A and 1B) on a 1.9kb HindIII fragment. The predicted amino acid sequence (SEQ ID NO:2) of this clone is 55-61 % sequence identity with human MC-3 and MC-5 receptors, and 46-47% sequence identity with the human MC-1 and MC-2 (ACTH) receptor. The sequence of the receptor has also been deposited in Genbank, Accession no. NM_{—}005912.

### EXAMPLE 2

### Construction of a Recombinant Expression Construct, DNA Transfection and Functional Expression of the MC-4 Receptor Gene Product

In order to produce recombinant mammalian cells expressing the human MC4-R melanocortin receptor of Example 1, cDNA from the receptor was cloned into a mammalian expression construct, the resulting recombinant expression construct transfected into human 293 cells, and cell lines generated that expressed the melanocortin receptor proteins in cellular membranes at the cell surface.

The MC-4 receptor was cloned in a 1.9 kb HindIII genomic DNA fragment after PCR amplification of a lambda phage clone into pcDNAI/Neo (Invitrogen). This plasmid was stably introduced into human 293 cells by calcium phosphate co-precipitation using standard techniques, and plasmid-containing cells selected in G418 containing media.

Melanocortin receptor coupling to G-proteins in cells expressing the MC-4 melanocortin receptor was used analyze expression of the receptor in cell colonies transfected with the expression vectors described herein as follows. Cells (˜1×10⁶) were plated in 6-well dishes, washed once with DMEM containing 1% bovine serum albumin (BSA) and 0.5mM IBMX (a phosphodiesterase inhibitor), then incubated for 45 minutes at 37° C. with varying concentrations of the melanotropic peptides. Following hormone treatment, the cells were washed twice with phosphate buffered saline and intracellular cAMP extracted by lysing the cells with 1 ml of 60% ethanol. Intracellular cAMP concentrations were determined using an assay (Amersham) which measures the ability of cAMP to displace [8-³H] cAMP from a high affinity cAMP binding protein (see Gilman, 1970, Proc. Natl. Acad. Sci. USA 67: 305-312).

The MC-4 receptor was found to couple to adenylate cyclase activity having the following pattern of agonist affinity:

NDP-MSH>des-acetyl-α-MSH>/=ACTH₁₋₃₉>/=α-MSH>>γ₂-MSH=ACTH₄₋₁₀

whereas the synthetic ACTH₄₋₉ analogue ORG2766 showed no detectable binding to the MC-4 receptor. The results of adenylate cyclase activity assays are shown in FIG. 2. EC₅₀ values for each of the tested MC-4 receptor agonists are as shown in Table I:

TABLE I

| Agonist | Ec₅₀ |
|---|---|
| NDP-MSH | 1.1 × 10⁻¹¹M |
| desacetyl-αMSH | 4.9 × 10⁻¹⁰M |
| ACTH₁₋₃₉ | 6.8 × 10⁻¹⁰M |
| α-MSH | 1.5 × 10⁻⁹M |
| γ₂-MSH | >10⁻⁷M |
| ACTH₄₋₁₀ | >10⁻⁷ |

### EXAMPLE 3

### Melanocortin Analogue Binding to Mammalian MC4-R Melanocortin Receptors

Recombinant cells prepared as described above in Example 2 were used to characterize receptor binding of a melanocortin analog comprising cyclic lactam heptapeptides.

The melanocortin receptor analogue SHU9119 having the chemical structure:

Ac-Nle⁴-cyclo(Asp⁵, D-Nal(2)⁷, Lys¹⁰) αMSH-(4-10)-amide (SEQ ID No.: 5)

was prepared as described in Hruby et al. (1995, J. Med. Chem. 38: 3454-3461).

The analog was tested for melanocortin receptor binding using a colorimetric assay system developed by some of the instant inventors (Chen et al., 1995, Analyt. Biochem. 226: 349-354) as follows. A series of concatamers of the synthetic oligonucleotide:

5′-GAATTCGACGTCACAGTATGACGGCCATGG-3′ (SEQ ID No: 6)

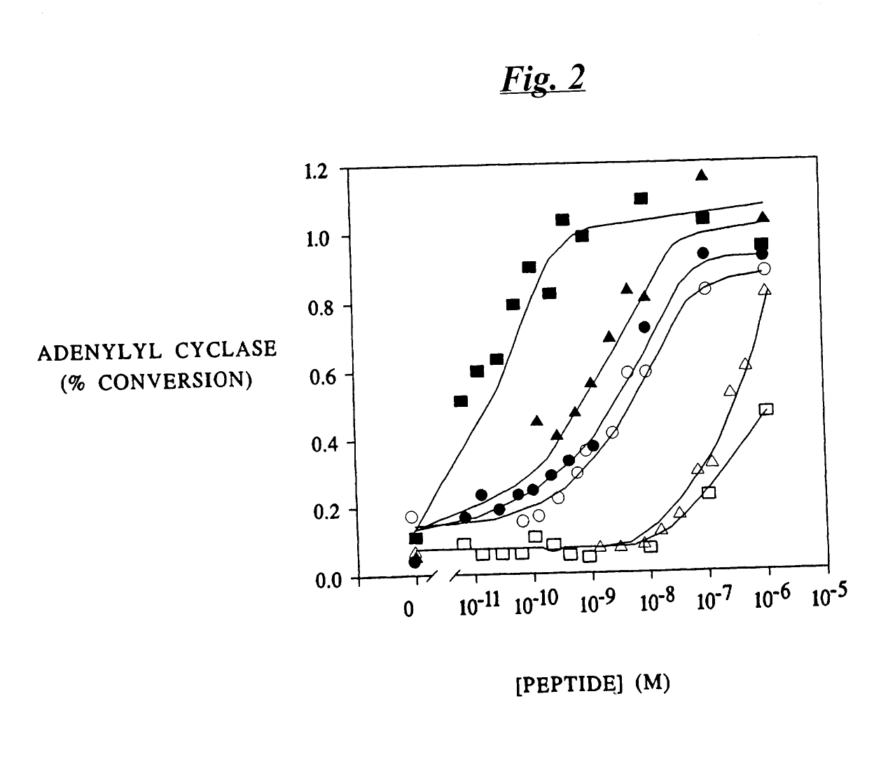

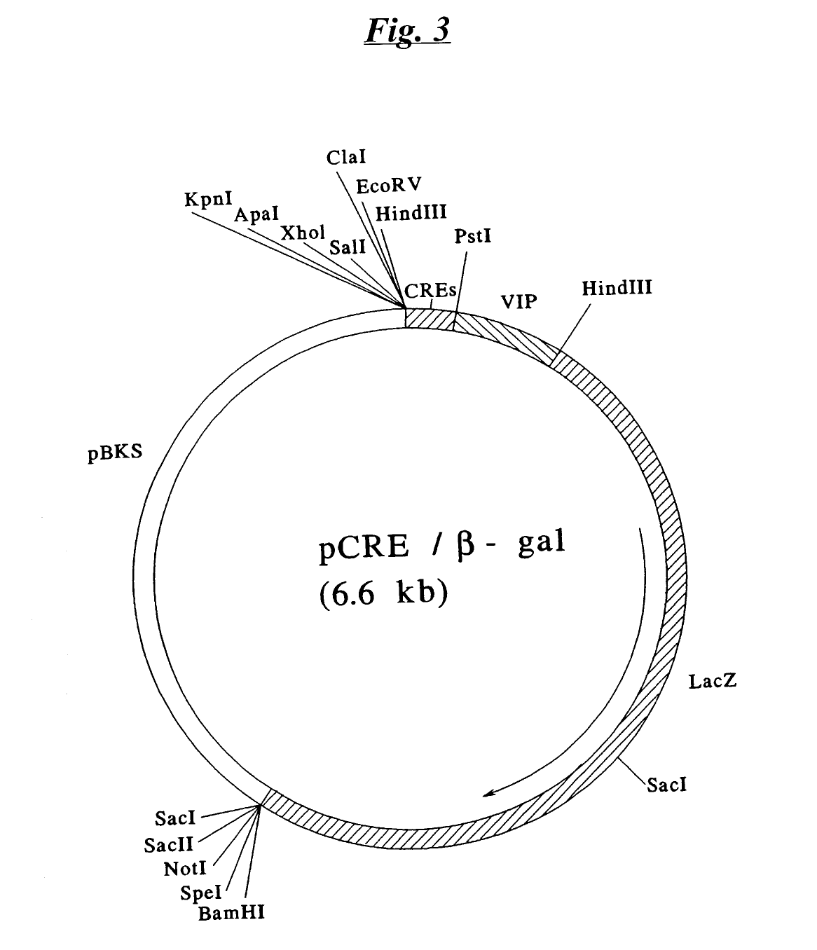

was produced by self-annealing and ligation and a tandem tetramer obtained. This fragment was cloned upstream of a fragment of the human vasoactive intestinal peptide (−93 to +152; see Fink et al., 1988, Proc. Natl. Acad. Sci. USA 85: 6662-6666). This promoter was then cloned upstream of the β-galactosidase gene from E. coli. The resulting plasmid construct is shown in FIG. 3.

Transient transfection of the pCRE/β-gal plasmid described above was performed as follows. MC-4 receptor expressing recombinant 293 cells were grown to between 40-60% confluency (corresponding to about 1.5 million cells/6 cm tissue culture plate), incubated with Opti-MEM (GIBCO-BRL, Long Island, N.Y.) and then contacted with a pCRE/β-gal-lipofectin complex prepared as follows. 3 μg plasmid DNA and 20 μL lipofectin reagent (GIBCO) were each diluted into 0.5 mL Opti-MEM media and then mixed together. This mixture was incubated at room temperature for 15-20 min., and then the mixture (1 mL) added to each 6 cm plate. Transfected plates were incubated at 37° C. for 5-24 h, after which the plates were washed and incubated with DMEM media (GIBCO) and the cells split equally into a 96-well culture plate.

To assay melanocortin receptor analogue binding, human 293 cells expressing the MC4-R melanocortin receptor was transiently transfected with pCRE/β-gal as described above and assayed as follows. Two days after transfection, cells were stimulated with hormones or hormone analogue by incubation for 6 h at 37° C. with a mixture comprising 10⁻¹²-10⁻⁶M hormone or analogue, 0.1 mg/mL bovine serum albumin and 0.1 mM isobutylmethylxanthine in DMEM. The effect of hormone or analogue binding was determined by β-galactosidase assay according to the method of Felgner et al. (1994, J. Biol. Chem. 269: 2550-2561). Briefly, media was aspirated from culture wells and 50 μL lysis buffer (0.25M Tris-HCl, pH 8/0.1% Triton-×100) added to each well. Cell lysis was enhanced by one round of freezing and thawing the cell/lysis buffer mixture. 10 μL aliquots were sampled from each well for protein determination using a commercially-available assay (BioRad, Hercules, Calif.). The remaining 40 μL from each well was diluted with 40 μL phosphate buffered saline/0.5% BSA and 150 μL substrate buffer (60 mM sodium phosphate/1 mM MgCl₂/10 mM KCl/5 mM β-mercaptoethanol/2 mg/mL o-nitrophenyl-β-D-galactopyranoside) added. Plates were incubated at 37° C. for 1 h and then absorbance at 405 nm determined using a 96-well plate reader (Molecular Devices, Sunnyvale, Calif.). A series of two-fold dilutions from 20 ng of purified β-galactosidase protein (Sigma Chemical Co, St. Louis, Mo.) were assayed in parallel in each experiment to enable conversion of OD₄₀₅, to known quantity of β-galactosidase protein.

The results of these experiments are shown in FIG. 4. This Figure shows the results of the β-galactosidase assay described above using cells expressing MC4-R and contacted with αMSH or a variety of αMSH analogues, including SHU9119. These results showed that SHU9119 had relatively weak agonist activity for human MC4-R receptors.

These results demonstrated the development of a colorimetric assay for cAMP accumulation as the result of melanocortin receptor binding to agonists and antagonists.

The action of SHU9119 and the endogenous mouse agouti peptide as agonists or antagonists of rodent MC receptors was first determined by examining their ability to elevate intracellular cAMP in 293 cell lines expressing mouse MC-4 melanocortin receptor (expressed as IC₅₀ values representing ligand concentration required for half-maximal inhibition of binding of (I-125)-(Nle⁴, D-Phe⁷)α-MSH tracer). Agonist/antagonist activity was also shown by demonstrating inhibition of cAMP elevation by the potent α-MSH analogue [Nle⁴, D-Phe⁷]α-MSH, using either a cAMP-responsive β-galactosidase reporter construct as described above, or by direct adenyl cyclase assay as described in Example 2 (wherein EC₅₀ values represent ligand concentration required for half-maximal activation of a cAMP-responsive β-galactosidase reporter). Competition binding experiments were determined as the amount of radioactivity bound in the presence of 5×10⁻⁶M unlabeled [Nle⁴, D-Phe⁷]α-MSH, and was typically 3-5% of total counts bound.

In these experiments, murine agouti peptide was produced using a baculovirus system as described by Lu et al. (1994, Nature 371: 799-802), with the modification that the agouti peptide was purified from baculovirus supernatants by 0.6M NaCl step elution from an EconoS cation exchange column (BioRad). Agouti peptide used in these assays was approximately 60% pure.

Competition binding assays were performed to determine whether SHU9119 had antagonist activity towards αMSH binding to MC-4 melanocortin receptors. These assays were performed as follows. Human 293 cells (100,000 cells/well in 24-well plates) expressing MC-4 melanocortin receptor prepared as described above were incubated with a solution of 1 mg/mL BSA in PBS containing 100,000 cpm (3.1×10⁻¹⁰M [¹²⁵I](Nle⁴, D-Phe⁷)αMSH and varying concentrations of αMSH, (Nle⁴, D-Phe⁷)αMSH or SHU9119. Cells were incubated for 30min at 37° C., washed twice with PBS-BSA, lysed with 0.5 mL 0.5N NaOH, and counted using a γ-counter to quantitate the amount of bound [¹²⁵I](Nle⁴, D-Phe⁷)αMSH. Control experiments showed non-specific binding to occur at about 3-5% levels, and this was taken into account when analyzing the experimental results.

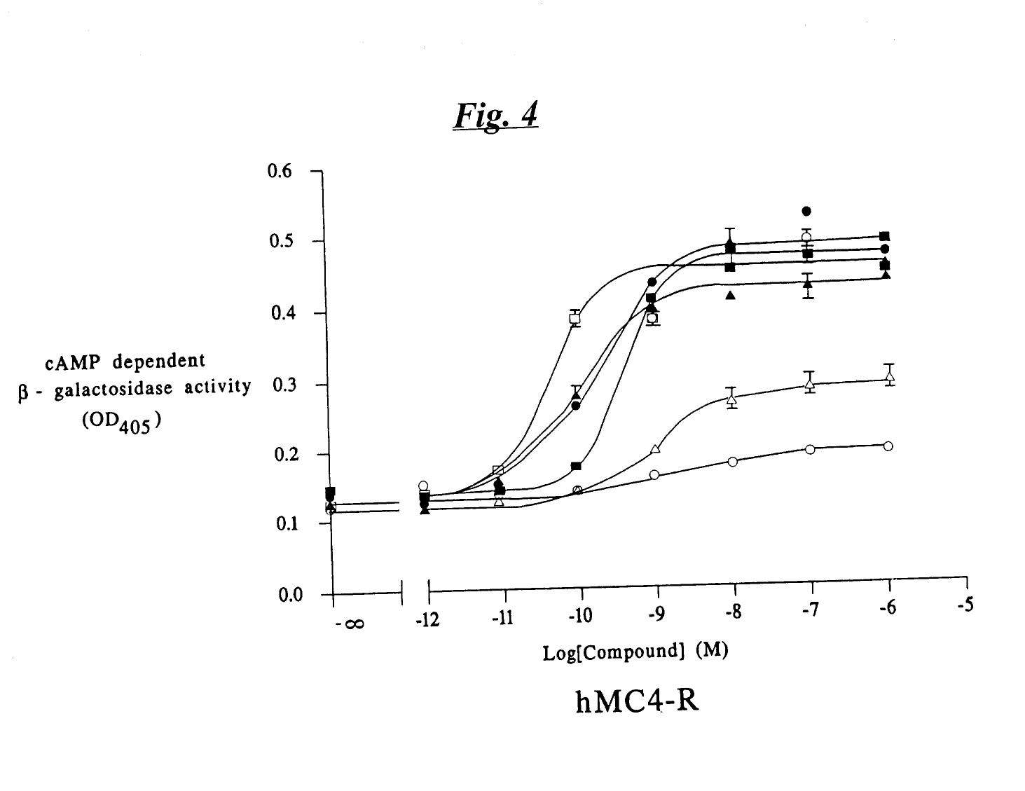

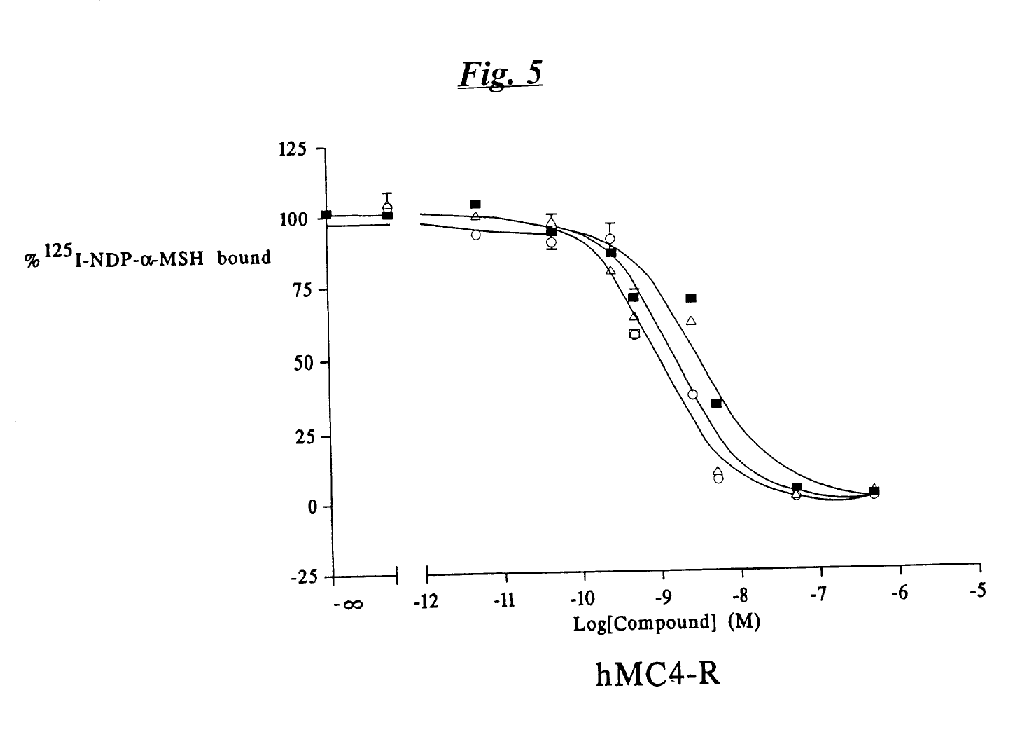

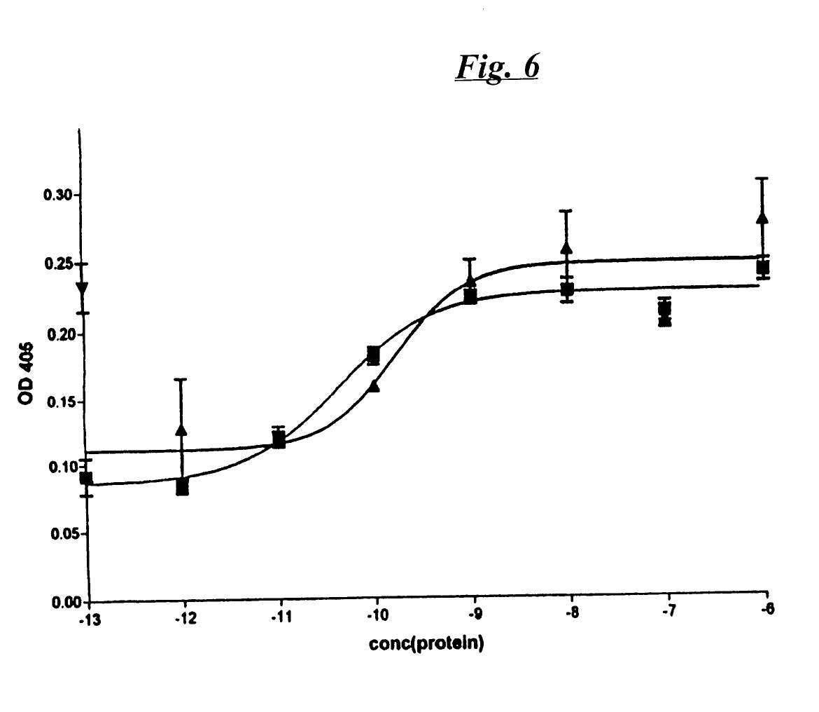

The SHU9119 analogue was found to be a potent antagonist of human MC-4 melanocortin receptors, as shown in FIG. 5. These assays showed pA₂ values of 8.3 and 9.3 for the human MC-4 melanocortin receptors, respectively, as determined using the method of Schild (1947, Brit. J Pharmacol. 2: 189-206). In contrast, no significant alteration in IC₅₀ values was detected in binding experiments using this analogue with MC-4 melanocortin receptors (FIG. 6).

Specific competition of α-MSH activation of human MC-4 melanocortin receptors by agouti was observed, although accurate IC₅₀ values could not be determined because the peptide preparation was not homogenous (results not shown). Specific competition of α-MSH activation of human MC4-R by agouti was previously disclosed (Lu et al., 1994, Nature 371: 799-802).

### EXAMPLE 4

### Use of MC Receptor-Expressing Recombinant Cells for Screening Compounds that Affect Feeding Behavior in Mammals

The results obtained in Example 3 indicated that cells expressing the MC-4 receptor were useful for characterizing compounds as MC-4 melanocortin receptor antagonists as a first step towards developing such antagonists for controlling feeding behavior in mammals, particularly anorexia, cachexia and other failure-to-thrive disorders.

Recombinant cells expressing mammalian, particularly human MC-4 melanocortin receptor protein were produced as described above in Example 3 wherein appropriate mammalian cells, such as human 293 cells, comprising a recombinant expression construct encoding the MC-4 receptor, wherein the cells functionally expressed MC-4 receptor protein.

The cells were used as follows. Receptor antagonist activity is assayed by transient or stable expression of a protein which produces a metabolite reporter molecule in response to receptor binding by MC-4 receptor proteins. An example of such a reporter system is the recombinant expression construct described in Example 4, wherein cAMP responsive elements (CREs) are operatively linked to a bacterially-derived β-galactosidase (β-gal) gene. In the event of receptor binding, cAMP is produced in the mammalian cell, and the CRE induces β-gal expression. When co-incubated with a colorless substrate for β-gal, receptor binding results in conversion of the colorless substrate to a blue-colored product, which can be easily scored visually or spectrophotometrically. Alternative reporter genes, such a luciferase, can also be used as reporter systems, provided that expression of the reporter molecule-producing protein is functionally linked to receptor binding of a test compound. Alternatively, cAMP production resulting from MC receptor binding can also be measured directly.

Antagonist activity is detected by inhibition of cAMP production by a standardized amount of a known receptor agonist, specific for the MC-4 receptor, assayed in the presence of a putative antagonist compound. Production of metabolite reporter molecules and their qualitative and quantitative detection is achieved as described herein above, and the specificity and potency of each antagonist compound characterized with regard to the degree of inhibition achieved for each receptor.

Assays are arranged so that antagonist activity can be identified, quantitated and correlated with expression of the MC-4 receptor. Automated assays using such cells are also envisioned, whereby the qualitative and quantitative detection of a reporter metabolite is detected in an array (such as a 96-well tissue culture plate) and the data collected and assembled into a computer data-base or other analytical program.

Compounds having antagonist activity with the MC-4 receptors detected using this assay are further screened in vivo to determine whether the observed receptor binding activity results in modification of feeding behavior when administered to an animal. In these assays, the MC-4 receptor binding compounds detected using the assay are administered intracranioventricularly as described below in Examples 5-8 to animals in which cachexia is experimentally induced. Feeding and locomotor activity is monitored in these animals, and compounds that inhibit or ameliorate cachexia are selected for further development.

In addition, systemic administration of compounds found to be active by ICV administration assays is used to screen such compounds for the ability to cross the blood-brain barrier. Such compounds are also useful as templates for modifications aimed at increasing the availability of these compounds in the brain after systemic administration, for increasing bioactivity, or both.

### EXAMPLE 5

### Preparation of Recombinant Targeting Vectors for Producing Mice Bearing a Homozygous Disruption of the MC4-R Gene Locus

The cloned human MC4-R gene disclosed in Example 1 above was used to prepare recombinant genetic constructs for producing mice bearing homozygous disruption of the MC4-R gene locus as follows.

The mouse MC4-R homolog of the human MC4-R gene described in Example 1 (Genbank Accession No. AF201662) was isolated from a genomic phage library prepared from 129/Sv mouse strain genomic DNA (Stratagene). PCR amplification of a portion of the human MC4-R DNA was performed as described in Example 1 to produce the probe, and hybridization was performed at high stringency (50% formamide, 1M NaCl, 50 nM Tris-HCl, pH 7.5, 0.1% sodiumpyrophosphate, 0.2% sodium dodecyl sulfate, 100 μg/ml salmon sperm DNA, 10×Denhardt's solution, 42° C.).

The “knock-out” construct was prepared in the vector pJN2 as follows. A 1.4 kb EcoRI-AvaI fragment of pBR322 was replaced with synthetic oligonucleotides having the sequence:

(SEQ ID NO.:7)

AATTAGCGCCCGCAGTATGCAAAAAAAAGCCCGCTCATTAGGCGGGCT
and

(SEQ ID NO.:8)

CCGAAGCCCGCCTAATGAGCGGGCTTTTTTTTGCATACTGCGGCCGCT.

The resulting plasmid, pJN1, was digested with NotI and the following oligonucleotides ligated to the NotI site to produce pJN2:

| | |
|---|---|
| GGCCGGCATGCATCAAGCTTATCTCGAGATCGTCGACTACCATGGTACATCGATCAGGTACCATCCCGGGGC and | (SEQ ID NO.:9) |
| GGCCGCCCCGGGATGGTACCTGATCGATGTACCATGGTAGTCGACGATCTCGAGATAAGCTTGATGCATGCC | (SEQ ID NO.:10) |

The pJN2 plasmid was digested with HindIII and SphI and a 1.2 kb HindIII-SphI fragment of mouse genomic DNA (extending from a HindIII site located about 600 bp 3′ from the mouse MC4-R sequence to a SphI site located about 1.8 kb 3′ from the mouse MC4-R sequence (thereby comprising the 3′ region of genomic homology) was subcloned into the vector. A 3.4 kb genomic DNA fragment (extending from anNcol site located approximately at the 5′ end of the mouse MC4-R sequence to a HindIII site located about 3.4 kb 5′ from the mouse MC4-R sequence (comprising the 5′ region of genomic homology) was also ligated into the plasmid, immediately 5′ of and in the same orientation as the 1.2 kb HindIII-SphI fragment. The PGK-neo expression cassette from the plasmid pKJ1 (as described in Tybulewicz et al., 1991, Cell 65: 1153-1163) containing the bacterial neo gene under the transcriptional control of the mouse phosphoglycerate kinase (PGK- 1) promoter and the PGK-1 polyA addition site was cloned in between the 5′ and 3′ genomic homology regions to generate the targeting vector.

This vector was linearized by digestion with NotI before use.

### EXAMPLE 6

### Use of Recombinant Targeting Vectors for Producing Mice Bearing a Homozygous Disruption of the MC4-R Gene Locus

Mice bearing a homozygous disruption of the MC4-R gene locus (“MC4-R knockout mice”) were produced substantially as described in Huszar et al. (1997, Cell 88: 131-141, incorporated by reference). The protocol is briefly set forth below.

Transfection of ES Cells and Blastocyst Injection

The targeting vector prepared as described in Example 4 above was introduced into mouse ES cells by electroporation as follows. An ES cell line (RF-8, as described in Huang et al., 1996, J. Cell. Biol. 133: 921-928, incorporated by reference herein) were grown on SNL76/7 mitotically inactive feeder cells. For electroporation, cells were trypsinized and resuspended at a concentration of about 1.1×10⁷ cells/mL in calcium and magnesium-free phoshate buffered saline (PBS; obtained from GIBCO, Gathersburg, Md.). Ten million cells in 0.9 mL were mixed with 20 μg of the linearized pJN2 targeting vector and subjected to electroporation using a pulse at 250V, 500 μF (Gene Pulser, BioRad). After electroporation, aliquots of 10,000 to 20,000 cells were plated on 100 mm culture plates containing a monolayer of SNL76/7 mitotically inactive feeder cells. The following day, the cells were selected with G418 (400-1000 μg/mL; GIBCO). Individual colonies were identified one week after selection, and divided into one well each of 96 well plates as described by Ramirez-Solis et al. (1993, Meth. Enzymol. 225: 855-878, incorporated by reference). DNA from individual clones was screened by Southern hybridization for homologous recombinants as described in Huszar et al. (ibid.).

ES cell clones having a Southern blot hybridization pattern indicating that at least one allele of the endogenous mouse MC4-R receptor gene had been disrupted by the targeting vector injected into C57B1/6J blastocysts as described in Bradley (1987, TERATOCARCINOMAS AND EMBRYONIC STEM CELLS, Robertson, ed., IRL Press: Oxford, UK, PP. 113-151, incorporated by reference) and implanted into pseudopregnant mice. Male chimeric mice were bred with C57B1/6J females, and agouti offspring (which represented germline transmission of the ES genome) were screened by Southern hybridization for the presence of the disrupted MC4-R gene. Heterozygous offspring were bred and homozygous offspring produced thereby identified by Southern hybridization.

### EXAMPLE 7

### Effects of MC4-R Antagonists on Lipopolysaccharide-induced Cachexia

The effect of MC4-R antagonists on lipopolysaccharide-induced cachexia was determined as follows. MC4-RKO mice and their wild-type (WT) controls were derived from the original C57BL/6J×129 colony (34) maintained within the Vollum Institute that had been bred five generations into the C57BL/6J strain. All mice were raised group housed in a 12 h light/dark cycle. Mice were weaned at 21 d and allowed ad libitum access to powdered Laboratory Rodent Diet (Purina) which was weighed and replaced daily. For studies measuring food intake, mice were housed individually and food intake estimated by measuring the weight of powdered food remaining in feeding chambers designed to maximize spill capture. To minimize error due to loss of food particles, all bedding was screened before and after the experiment to capture any spilled food. Food remaining in the feeding chamber was also screened to remove any bedding or other debris. C57B1/6J mice (25-33 g, Jackson Labs) were housed and fed similarly. All studies were conducted according to the NIH Guide for the Care and Use of Laboratory Animal and approved by the Animal Care and Use Committee of the Oregon Health Sciences University.

Cannula Placement.

C57B1/6J mice were anesthetized with halothane and placed in a stereotaxic apparatus (CARTESIAN Research, Inc.). A sterile guide cannula with obdurator stylet was stereotaxically implanted for intracerebroventricular (icv) injection with the coordinates of 0.5 mm posterior to the bregma, 1-1.6 mm lateral to the midline and 2 mm blow the bregma. The cannulla was then fixed in place using dental cement. The animals were housed separately after surgery at least one week for recovery before experiments. The positions of the cannulae were verified at the end of experiments by histological analysis; in animals in which cerebrospinal fluid (CSF) return was not obvious, the position of the cannulae were tested by dye administration prior to sacrifice.

AGRP and LPS Administration.

Each animal was handled daily for a minimum of five consecutive days prior to the initiation of the experiment, simulating the restraint used during the injection of the compounds. Artificial cerebrospinal fluid (ACSF) or agouti-related peptide (AGRP; Phoenix Pharmaceutical Co., Mountain View, Calif.) diluted in ACSF was infused in a total volume of 2 mL over 30 seconds in lateral ventricle-cannulated mice. In the LPS experiments, LPS (E. Coli 055 :B5, Sigma Chemical Co.) was dissolved in normal saline and administered i.p. For the first LPS injection, male animals aged 6-7 weeks were used. In an identical repeat experiment, female animals, aged 5 weeks were used. In the tumor models, male animals, age 4 weeks at the start of the experiment were used. MC4-RKO mice and littermate controls had basal feeding monitored for two days, and then during each twelve hour period following an i.p. saline injection (1700 h) prior to injection of 100 μg/kg of LPS. In C57B1/6 WT animals, AGRP (84-132 amino acid fragment, Neurocrine, San Diego, Calif.) was administered at 1500 h, and 50 μg/kg LPS was administered at 1700 h. A second dose of 100 μg/Kg was given 60 hr after the first dose in the second experiment. No food was available between AGRP administration and LPS administration and 24 hr feeding was measured starting at 1700 h. In the tumor models, AGRP or ACSF was administered at 1400 h with each administration. AGRP administration prevents LPS-induced cachexia.

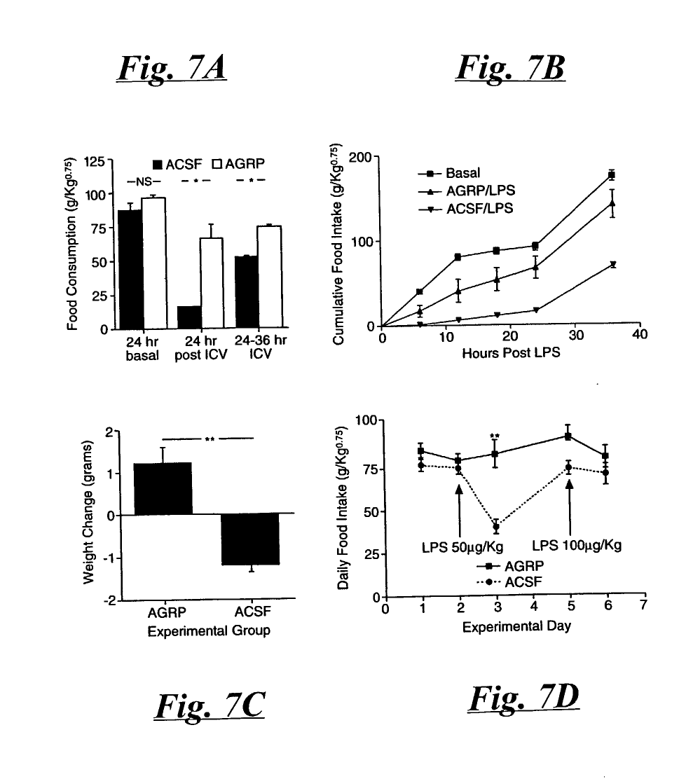

Basal feeding was measured every six hours in two age and sex-matched groups after simulated icv injection and intraperitoneal (i.p.) saline injection. Twenty-four hours later, AGRP was administered at 1500 h, and LPS was administered i.p. at 1700 h. Intracerebroventricular (icv) injection of the 84-132 fragment of AGRP (2.5 nmoles in 2 μL ACSF) prevented LPS (50 μg/kg)-induced decrease in feeding (FIG. 7A), even in the 24-36 hr period after LPS treatment. Feeding was measured every six hours for 24 hours, then every 12 hours for 48 more hours. The difference between feeding curves was significant when expressed both as weight normalized intake (n=6, p<0.001, FIG. 7B) or as a percent of basal feeding (n=6, p<0.001 vs. post-saline and sham icv injection, data not shown). Thereafter, the ACSF treated group demonstrated a recovery of normal feeding as expected (24 hour feeding from hour 36 to 58 post-LPS; 91±3 g/Kg^{0 75} AGRP vs 92±8 g/Kg^{0 75}ACSF; p=0.98). AGRP also prevented weight loss in this model of illness (n=6, p<0.000 1, FIG. 7C). In a second experiment, a second dose of LPS (100 μg/Kg) was given after recovery from the first dose three days after the first LPS injection (FIG. 7D). In this experiment, the ACSF-treated animals recovered to 95% of basal feeding on the third day after the first LPS injection after showing a significant drop in food intake relative to the AGRP group (n=7 AGRP, n=6 ACSF, p<0.0001). Interestingly, the AGRP treated animals continued to show a relative hyperphagia and consumed 118±6% of basal food intake on that third day (n=7, p<0.02). The second LPS injection did not result in a significant decrease in feeding in either group, demonstrating LPS tolerance in these animals (Langhans et al., 1990, Physiol. Behav.. 47: 805-813).

MC4R KO Mice Resist LPS-induced Cachexia and Illness Behavior.

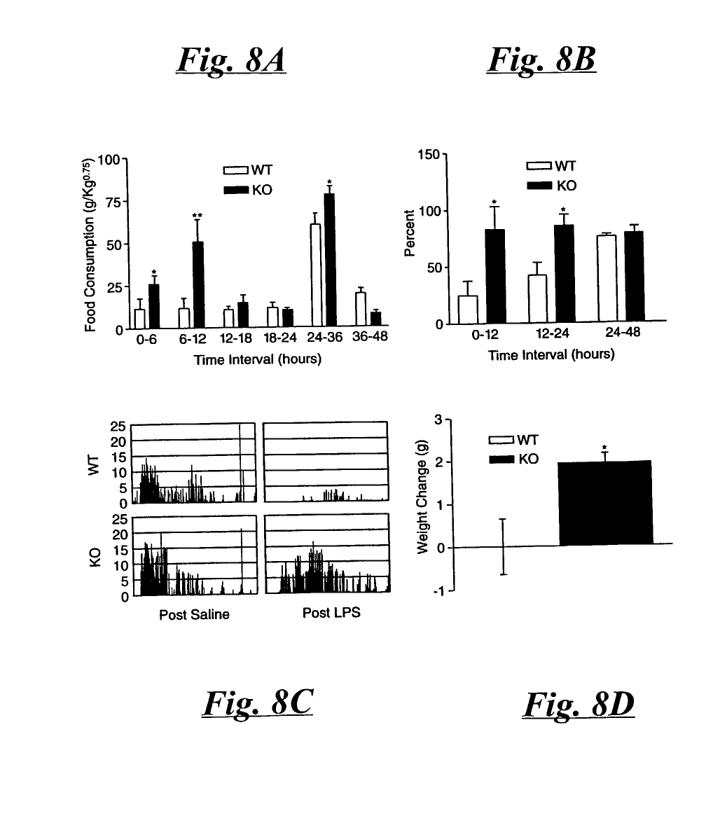

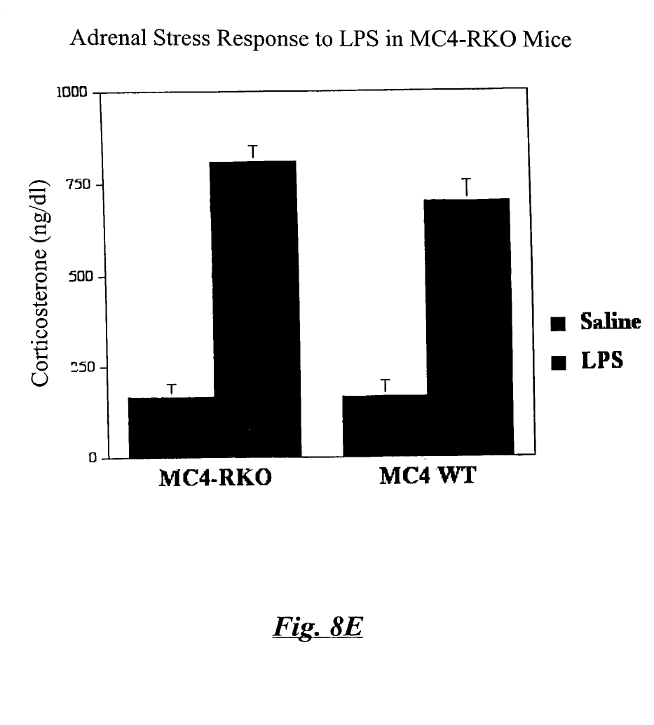

MC4-RKO mice were tested for their response to LPS injections. Parameters monitored included food and water intake, lick counts, wheel running activity, and weight gain. Six week old male MC4-RKO mice were slightly but not significantly heavier than their wild-type littermates (KO 17.5±0.8 vs. WT 15.7±0.4, p=0.07) and all feeding data was normalized to weight. Basal feeding after i.p. saline was not different between groups (n=5, p=0.8). LPS administration resulted in a significant decrease in feeding in the WT animals which was apparent for 36 hours after injection (FIG. 8A). This decrease in intake was not seen in MC4-RKO animals (n=5, p<0.01 vs. WT) when measured either as total food intake or as a percentage of basal intake after saline injection (FIG. 8B). Water intake as a percentage of basal was also greater in MC4-RKO mice but the difference was not significant (MC4-RKO 70% ±12% vs. WT 46%±14%, p>0.05). However, total lick counts were significantly different after LPS (MC4-RKO 1.8±0.4 counts/min vs WT 0.4 ±0.09 counts/min, p<0.05). Wheel running activity was similar between groups after saline injection, but greatly decreased in the WT animals after LPS injection (FIG. 8C). Twenty-four hour total number of turns per minute were similar after saline injection (MC4-RKO 2.5±0.6 turns/min vs WT 2.8±0.8 turns/min, p=0.8), while after LPS injection the WT animals showed a decrease in total 24 hour turns while MC4-RKO animals maintained normal activity (MC4-RKO 2.4 ±0.9 turns/min vs WT 0.1±0.05 turns/min, p<0.05). WT animals failed to gain weight during the 60 hr experimental period while MC4-RKO mice continued to gain weight (FIG. 8D, p<0.05).

The MC4-R knockout did have a normal response to LPS in terms of its “stress axis”. There was a normal release of cortisol in response to LPS (FIG. 8E). The MC4-R knockout also showed resistance to wasting of lean body mass during tumor growth. This is significant, because it indicates that the animal is not simply maintaining weight by retaining water or fat, an outcome with little clinical benefit. In addition, the animals used in the study continued to grow normally while wildtype animals did not.

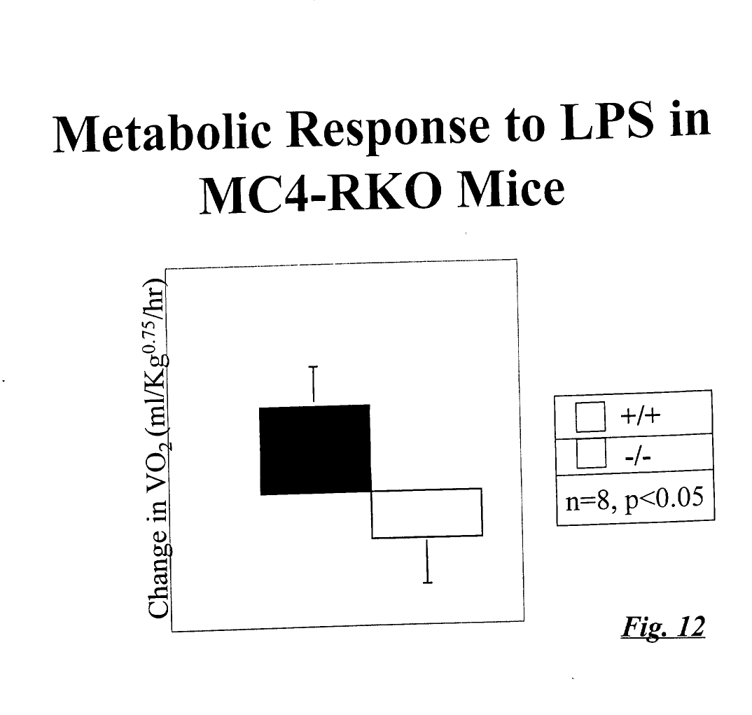

Energy metabolism was assayed by indirect calorimetry measuring VO₂ for the amount of oxygen used for energy production in the mice. These results are shown in FIG. 12. LPS-treated wildtype mice showed an increase in VO₂ of about 150 mL/kg^{0.75}/hr, while LPS-treated knockout mice showed a decrease in VO₂ of about 75 mL/kg^{0 75/}hr. These differences were also statistically significant (p <0.05). These results demonstrated that the knockout mice resisted the increase in metabolic rate that normally accompanies LPS-induced cachexia.

These results demonstrate that ablation of MC-4 receptor activity in the MC-4R knockout mice inhibited the cachexigenic effects of LPS administration, and suggested that inhibition of MC-4 receptor by MC-4 receptor antagonists can inhibit or ameliorate cachexia by preventing cachexia-associated disregulation of energy metabolism mediated through the MC-4 receptor.

### EXAMPLE 8

### Effects of MC4-R Antagonists on Tumor-Induced Cachexia

Tumor Models

Lewis lung carcinoma (LLC) cells and Englebreth-Holm-Swarm Sarcoma (EHS) tumors were maintained either as a primary culture in DMEM with 10% fetal bovine serum or in vivo, respectively, as recommended by the supplier (American Type Culture Collection, Manassas, Va.). LLC tumor cells were harvested during exponential growth of the culture, washed in Hanks balanced salt solution, and 1×10⁶ cells were injected subcutaneously into the upper flank of the mice. EHS sarcoma tissue was dissected from a donor animal, and an approximately 3 mm cube of tissue was implanted subcutaneously above the rear flank. Sham operated animals received an implant of a similar amount of donor muscle tissue. In all cases, the time of appearance of a tumor mass was noted, and all animals were found to have a palpable tumor within four (LLC) or eight (EHS) days of the start of the experiment. At the time of sacrifice, tumors were dissected away from surrounding tissue and weighed. Gross examination of all organs did not reveal the presence of any observable metastasis. Trunk blood was collected at the time of sacrifice for measurement of serum leptin with a rat leptin radioimmunoassay kit (Linco Research, Inc., Manassas, Va.).

Motor Activity, Feeding Activity.

Animals were housed individually in metabolic cages equipped with a running wheel (Mini-Mitter Co., Sunriver, Oreg.). The metabolic cages usage allowed telemetric monitoring of circadian rhythms as assessed via multiple physiological parameters. The wheel revolutions were quantified by recording the magnetic switch closures of a magnet placed on the revolving wheel. For feeding recordings, feeding counts and duration were recorded when the animals interrupted an infra-red beam above the feeding chambers.

Statistical Methods.

Differences between feeding, activity, and water consumption curves in all experiments were analyzed by two-way, repeated measures ANOVA with time and treatment as the measured variables. Final tumor and body weights were analyzed by Student's t-test when two groups were included, or one way ANOVA with post-hoc analysis when three groups were included. Data sets were analyzed for statistical significance using either the PRISM software package (GraphPad) for ANOVA with repeated measures, or in EXCEL (Microsoft) using Student's t-test.

Effect of AGRP Administration in C57B1/6J Mice Bearing a Syngenic Sarcoma.

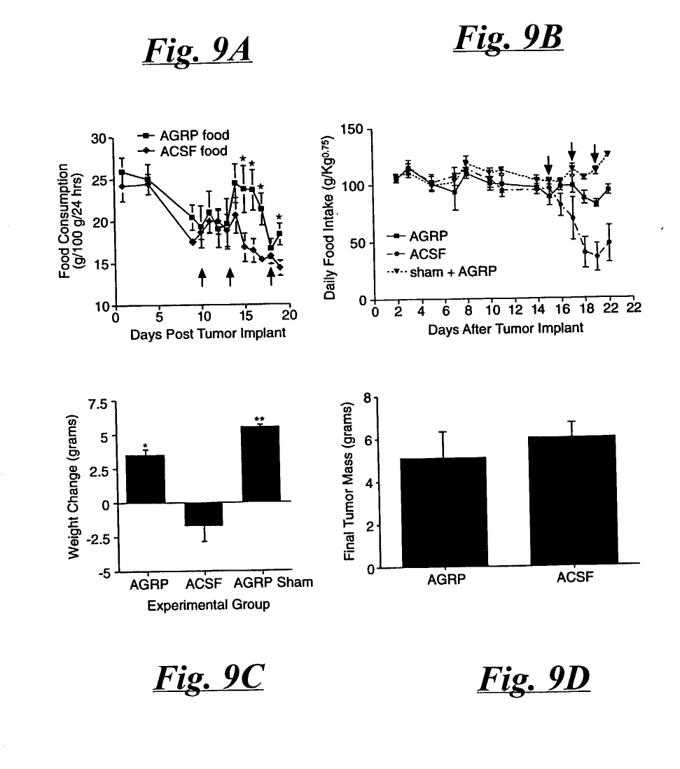

The effects of AGRP administration in animals with hypophagia and weight loss due to the presence of a growing sarcoma were examined. In an initial experiment, daily food intake and weight was followed until the tumor-bearing animals had food intake that was 75-80% of basal for three consecutive days. This occurred on day 12 post-implant, on average four days after a palpable tumor was present. Intracerebroventricular (icv) injection of the 84-132 fragment of AGRP (2.5 nmoles in 2 μL ACSF) caused a return to basal feeding levels in the treated group within 48 hours of injection (FIG. 9A, AGRP injected 96%±5% on day 14). However, the difference between treatment groups was not significant on that day (n=5, p=0.2). A second injection on day 14 post implant sustained this normalization of food intake, while the ACSF treated animals continued to have gradually decreasing intake. This effect lasted for three days, with a return to the relatively anorexic state seen by day 18 post implant. A third injection of AGRP on this day again raised the food intake of AGRP treated animals with both groups sacrificed on day 19 due to the growth of the tumor. Overall ANOVA for feeding in this study was significant (n=5, p<0.0003), with post hoc testing significant on days 16, 17, and 19 post tumor implant. AGRP treatment had no effect on final tumor mass (AGRP 5.1±1.3 g vs ACSF 6.0±0.8 g, p=0.6), but did prevent the weight loss observed in the ACSF treated animals (AGRP +2.1±0.8 g vs ACSF −0.21±0.03 g, p<0.05).

In a second experiment the ability of AGRP to prevent the onset of cachexia and maintain normal feeding and growth was tested. Animals were examined daily for the presence of a palpable tumor, with all animals having tumors by day 14 post implantation, and none prior to day 12. Animals were then injected with AGRP (2.5 nmoles in 2 μL ACSF) or ACSF every 48 hours until sacrifice. A sham-tumor implanted group was included for comparison and was also given AGRP. AGRP administration prevented the tumor-induced decline in food intake in the AGRP treated animals and resulted in a relative hyperphagia in the sham tumor animals (FIG. 9B, p<0.0001). Two animals were removed from the ACSF group 24 hours prior to the end of the experiment due to moribund appearance. These animals had eaten less than 20% of basal during the 24 hours prior to sacrifice. AGRP treatment did not affect final tumor mass (FIG. 9D, p=0.5) but did prevent the tumor-induced weight loss (FIG. 9C, p<0.0001). Post-mortem dissection did not reveal the presence of any discernible subcutaneous, epididymal, or visceral fat pads in any experimental group.

Resistance to Cachexia in MC4-RKO Mice Bearing a Syngenic Adenocarcinoma.

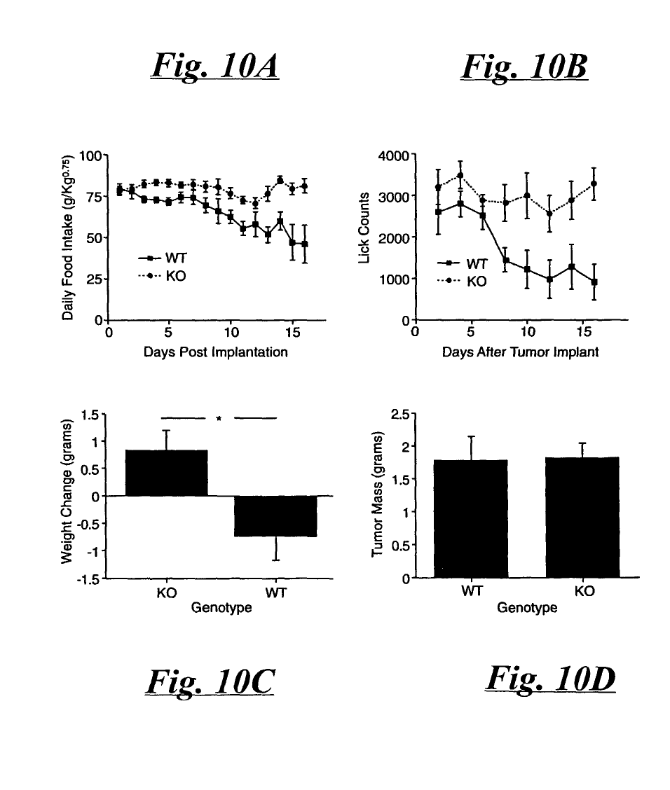

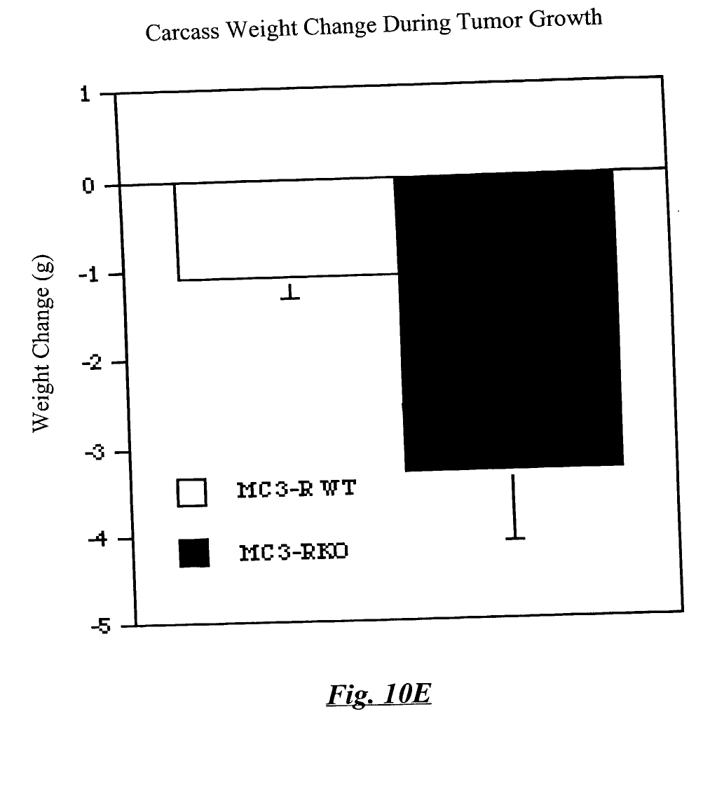

To confirm and extend the findings in the sarcoma model, we next tested the response of MC4-RKO mice to the growth of a cachexigenic adenocarcinoma (Matthys et al, 1991, Eur. J. Cancer27:182-187; Llovera et al.,1998, Cancer Lett. 130:19-27). Parameters monitored included food and water intake, lick counts, meal frequency and duration, wheel running activity, weight gain, and tumor mass. WT control mice began to show decreased 24 hour feeding at day 3 post tumor implantation, prior to the presence of a palpable tumor (FIG. 10A). The overall feeding curves were noticeably different from that day onward, with the WT animals consuming 56% of MC4-RKO levels by the final day of the experiment (FIG. 10A, n=5, p<0.0⁰01). The change in water lick counts paralleled the change in food consumption (FIG. 10B, p<0.0001). Wild type animals showed a prompt decline in wheel running activity to 30%±10% of basal activity by day seven post implantation, while MC4-RKO mice showed a slower decline (day 7, 70%±14% p<0.05). However, the MC4-KO animals eventually decreased their running activity as well (66%±18% of basal on the final day), and the overall activity curves were not different between groups (n=5, p=0.09). MC4-RKO animals gained carcass weight while control animals lost weight (FIG. 10C, n=5, p<0.05). The final tumor mass was not different between groups (FIG. 10D, p=0.9). Serum leptin levels were also not different between groups (WT 2.3±1.4 ng/mL vs. KO 2.2±1.1 ng/mL, p=0.98). A repeat trial of this experiment revealed similar results with significant differences observed in carcass weight change (WT −0.3±0.5 g vs. KO 1.4±0.4 g, p<0.05), but not in final tumor mass (WT 1.7±0.3 g vs. KO 1.4±0.4 g, p=0.61). Preliminary carcass analysis indicates that the majority of the difference between groups is due to a larger amount of lean body tissue in the MC4-RKO animals.

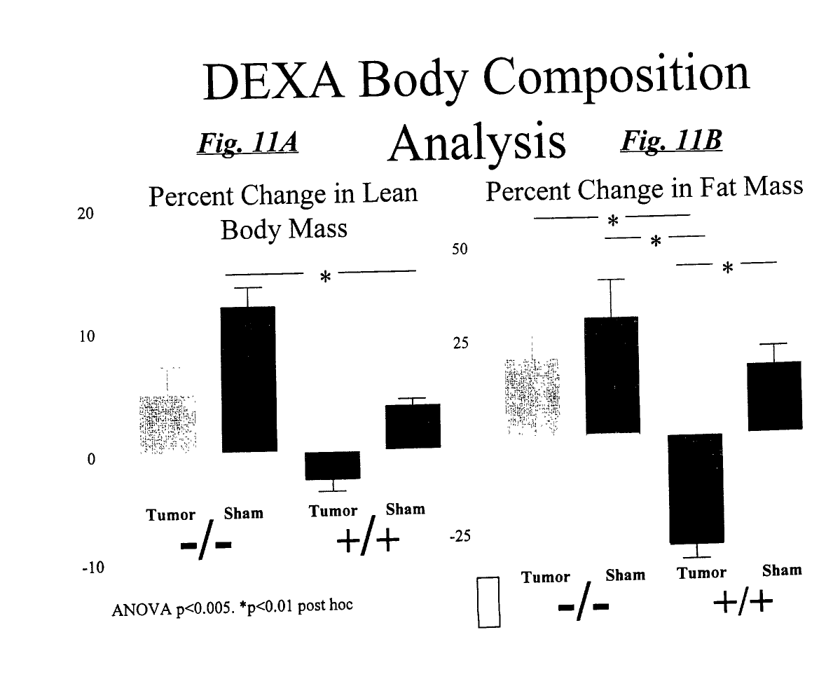

Body composition analysis was performed to determine whether the reduction in weight loss determined for MC-4 melanocortin receptor antagonist-treated cachexic animals was due to a reduction in lean body mass loss or was merely the consequence of nonsignificant changes in body mass (such as increased water retention). In these experiments, dual X-ray absorptiometry (DEXA) body composition analysis was used to determine the percentage change in lean body mass (FIG. 11A) and fat mass (FIG. 11B) in tumor-bearing wildtype (+/+) and MC-4RKO (−/−) mice. As shown in FIG. 11A, both wildtype (+4±0.6%) and knockout (+12±2%) mice showed an increase in lean body mass when injected with saline (sham) rather than with tumor cells. In contrast, wildtype mice showed a reduction (−2±1 %) in lean body mass when injected with tumor cells, but knockout mice showed an increase (+5±3%) in lean body mass that was only slightly less than the increase seen in the sham injected KO mice. Similar results were seen in body fat mass (FIG. 11B), but the reduction in fat mass in wildtype mice (−29±4%) bearing tumor cells was much greater than with lean body mass, and the corresponding difference in body fat mass between tumor cell-bearing wildtype and knockout (+21±7%) mice was accordingly much greater. Sham injected wildtype (+18±5%) mice had a lower body fat mass than knockout (+31±10%) mice. These results were assessed by ANOVA and were significant (p<0.005).

In parallel experiments using melanocortin MC-3 knockout mice (produced analogously to the MC-4 receptor knockout mice described above), the MC-3 knockout mice were more susceptible to cancer cachexia (see FIG. 10E), and had greater weight loss in these animals during tumor growth. Thus, the effect of AGRP (an MC3 and MC4 antagonist) to block cachexia is due entirely to its effects on the MC4-R, not on the MC3-R. Indeed, drugs with specific MC4 activity would be expected to be superior to AGRP in this regard.

These results demonstrate that cachexia induced by lipopolysaccharide (LPS) administration and by tumor growth is ameliorated by central MC4-R blockade. Both genetic and pharmacologic blockade of central MC4-R signaling can prevent the hypophagia, bypodipsia, and decreased locomotor activity seen after induction of a complex and pleotropic cytokine response. MC4-RKO mice or mice administered the MC3 -R/MC4-R antagonist AGRP resisted tumor-induced loss of lean body mass, and maintain normal circadian activity patterns during tumor growth. The final tumor mass is not affected in these animals, providing further support for the role of MC4-R antagonism in the treatment of cachexia in disease states.

It should be understood that the foregoing disclosure emphasizes certain specific embodiments of the invention and that all modifications or alternatives equivalent thereto are within the spirit and scope of the invention as set forth in the appended claims.

What we claim is:

1. A method for characterizing a compound that prevents, inhibits or ameliorates cachexia in an animal, the method comprising the steps of:
(a) administering the compound to an animal with cachexia;
(b) comparing energy metabolism, food intake or motor activity in said animal with energy metabolism, lean body mass, food intake or motor activity in an animal with cachexia that is not administered the compound; and
(c) identifying compounds that prevent, inhibit or ameliorate cachexia when less fat or lean body mass loss or reduction in energy metabolism, food intake or motor activity is detected in animals to which the compound has been administered than in animals to which the compound has not been administered.

2. The method of claim 1, wherein cachexia is experimentally induced in the animal

3. The method of claim 2, wherein cachexia is experimentally induced in the animal by administration of lipopolysaccharide or implantation of tumor cells or tissue.

4. The method of claim 1, wherein the compound is an MC-4 melanocortin receptor inhibitor.

5. A method of preventing or treating a pathological feeding behavior in an animal, the method comprising administering an effective amount of a mammalian melanocortin MC-4 receptor antagonist to the animal.

6. The method of claim 5, wherein the pathological feeding behavior is cachexia.

7. A method for characterizing a compound that prevents, inhibits or ameliorates cachexia in an animal and that is an antagonist of a mammalian MC4-R melanocortin receptor, the method comprising the steps of:
(a) providing a mammalian cell comprising a recombinant expression construct encoding a mammalian melanocortin receptor that is the MC-4 melanocortin receptor, wherein the mammalian cell expresses the melanocortin receptor encoded by the recombinant expression construct, or cell membranes prepared therefrom;
(b) contacting the cells or membranes prepared therefrom with an amount of an MC-4 melanocortin receptor agonist sufficient to produce a detectable amount of a metabolite produced in the cells that bind the agonist, in the presence and absence of a test compound to be characterized as an antagonist of the MC-4 melanocortin receptor;
(c) assaying the cells or membranes prepared therefrom for agonist binding or for the metabolite produced in cells that bind an MC-4 melanocortin receptor agonist;
(d) comparing agonist binding or metabolite production in the cells in the presence of the test compound to metabolite production in the cells in the absence of the test compound;
(e) identifying the compound as an MC-4 melanocortin receptor antagonist when less metabolite is produced in the presence of the compound than in the absence of the compound or when less agonist is bound in the presence of the compound than in the absence of the compound;
(f) administering the compound to an animal that expresses MC-4 receptor and in which cachexia is experimentally induced;
(g) comparing energy metabolism, food intake or motor activity in said animal with energy metabolism, lean body mass, food intake or motor activity in an animal in which cachexia is experimentally induced and to which the compound is not administered; and
(h) identifying the compound as an MC-4 melanocortin receptor that prevents, inhibits or ameliorates cachexia when less fat or lean body mass loss or reduction in energy metabolism, food intake or motor activity is detected in animals to which the compound has been administered than in animals to which the compound has not been administered.

8. The method of claim 7, wherein the metabolite detected in subpart (c) is cyclic AMP.

9. The method of claim 7, each of the cells further comprising a recombinant expression construct encoding a cyclic AMP responsive element (CRE) transcription factor binding site operatively linked to a nucleic acid sequence encoding a protein capable of producing a detectable metabolite.

10. The method of claim 9, wherein the nucleic acid sequence encodes β-galactosidase.

11. The method of claim 9, wherein the recombinant expression construct is pCRE/β-galactosidase.

12. The method of claim 9, wherein the detectable metabolite produced by the protein encoded by the recombinant expression construct is produced by binding of the test compound to the mammalian MC4-R melanocortin receptor.

13. A method according to claim 7, wherein the MC-4 receptor is a human MC-4 receptor having an amino acid sequence identified as SEQ ID NO: 2.

14. The method of claim 7, wherein the MC-4 melanocortin receptor agonist is detectably-labeled.

15. The method of claim 14 wherein the MC-4 melanocortin receptor agonist is detectably labeled with a radioisotope, a fluorescent label, a hapten, an enzymatic label or an antigenic label.


SEQUENCE LISTING (10 sequences)

SEQ ID NO: 1 - DNA, 1671 bp, Homo sapiens
  CDS (394)..(1389)
agcttccgagaggcagccgatgtgagcatgtgcgcacagattcgtctcccaatggcatggcagcttcaaggaaaattattttgaacagacttgaatgcataagattaaagttaaagcagaagtgagaacaagaaagcaaagagcagactctttcaactgagaatgaatattttgaagcccaagattttaacgtgatgatgattagagtcgtacctaaaagagactaaaaactccatgtcaagctctggacttgtgacatttactcacagcaggcatggcaattttagcctcacaactttcagacagataaagacttggaggaaataactgagacgactccctgacccaggaggttaaatcaattcagggggacactggaattctcctgccagcatggtgaactccacccaccgtgggatgcacacttctctgcacctctggaaccgcagcagttacagactgcacagcaatgccagtgagtcccttggaaaaggctactctgatggagggtgctacgcgcaactttttgtctctcctgaggtgtttgtgactctgggtgtgatcagcttgttggagaatatcttagagattgtggcaatagccaagaacaagaatctgcattcacccatgtactttttcatctgcagcttggctgtggctgatatgctggtgagcgtttcaaatggatcagaaaccattatcatcaccctattaaaccgtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcagcataagttgtatctgggcagcttgcacggtttcaggtattttgttcatcatttactcagatagtagtgctgtcatcatctgcctcatcaccatgttcttcaccatgctggctctcatggcttctctctatgtccacctgttcctgatggccaggcttcacattaagaggattgctgtcctccccggcactggtgccatccgccaaggtgccaatatgaagggagcgattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctcttcctatcccctgggaggcctttgtgacttgtctagcagatattaaatggggacagagcacgcaatataggaacatccataagagactttttcactcttaccctacctgaatattctacttctgcaacagctttctcttccgtgtagggtactggttgagatatccattgtgtaaatttaagcctatgatttttaatgagaaaaaatgcccagtctctgtattatttccaatctcatgctacttttttggccataaaatatgaatctatgttataggttgtaggcactgtggatttacaaaaagaaaagtccttattaaaagatt

SEQ ID NO: 2 - protein, 332 aa, Homo sapiens
MVNSTHRGMHTSLHLWNRSSYRLHSNASESLGKGYSDGGCYAQLFVSPEVFVTLGVISLLENILEIVAIAKNKNLHSPMYFFICSLAVADMLVSVSNGSETIIITLLNRTDTDAQSFTVNIDNVIDSVICSSLLASICSLLSIAVDRYFTIFYALQYHNIMTVKRVGISISCIWAACTVSGILFIIYSDSSAVIICLITMFFTMLALMASLYVHLFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIISSYPLGGLCDLSSRY

SEQ ID NO: 3 - DNA, 35 bp, Artificial Sequence
  Description of Artificial Sequence degenerate oligonucleotide primer
gagtcgacctgtgygysatyrcnntkgacmgstac

SEQ ID NO: 4 - DNA, 32 bp, Artificial Sequence
  Description of Artificial Sequence degenerate oligonucleotide primer
cagaattcagwagggcanccagcagasrygaa

SEQ ID NO: 5 - protein, 7 aa, Artificial Sequence
  SITE (1): “Xaa” is norleucine
XDHXRWK

SEQ ID NO: 6 - DNA, 30 bp, Artificial Sequence
  Description of Artificial Sequence degenerate oligonucleotide primer
gaattcgacgtcacagtatgacggccatgg

SEQ ID NO: 7 - DNA, 48 bp, Artificial Sequence
  Description of Artificial Sequence oligonucleotide primer
aattagcgcccgcagtatgcaaaaaaaagcccgctcattaggcgggct

SEQ ID NO: 8 - DNA, 48 bp, Artificial Sequence
  Description of Artificial Sequence oligonucleotide primer
ccgaagcccgcctaatgagcgggcttttttttgcatactgcggccgct

SEQ ID NO: 9 - DNA, 72 bp, Artificial Sequence
  Description of Artificial Sequence oligonucleotide primer
ggccggcatgcatcaagcttatctcgagatcgtcgactaccatggtacatcgatcaggtaccatcccggggc

SEQ ID NO: 10 - DNA, 72 bp, Artificial Sequence
  Description of Artificial Sequence oligonucleotide primer
ggccgccccgggatggtacctgatcgatgtaccatggtagtcgacgatctcgagataagcttgatgcatgcc